SEQUENCE LISTING (118 sequences)

SEQ ID NO: 1 - protein, 60 aa, Conus striatus
KDRPSLCDLPADSGSGTKAEKRIYYNSARKQCLRFDYTGQGGNENNFRRTYDCQRTCLYT

SEQ ID NO: 2 - protein, 52 aa, Conus striatus
GRPKDRPSYCNLPADSGSGTKPEQRIYYNSAKKQCVTFTYNGKGGNGNNFSR

SEQ ID NO: 3 - protein, 59 aa, Conus magus
RPSVCNKPADKGPCAGSEKRFYFSTYHNECRTFKYGGCEGNGNNFIHVYNCRRTCVYPM

SEQ ID NO: 4 - DNA, 558 bp, Conus magus
  CDS (231)..(494)
gaattcgcccttgctgatggcgatgaatgaacactgcgtttgctggctttgatgaaaagtccacgtcagcagagggagagagagagaccttaggagctgtgtattctccagtcatctgacggccctatccacgtcagtgctgtttcgtctgtcagccagtccacatcaccgctcaaccatctgtcagcctgtttacgtcagcactgtcatctgactgtccaggttaaaaaatggagggacgtcgttttgctgctgttctgatcctgaccctctgtatgcttgcatctggggctgtcgctgcgagacctaaggatcgaccgagttattgcaatctaccagcggacagtgggtcgggcacaaagcctgagcagagaatttactacaatagcgctcgagaacagtgtttaacgttcacttacaatggaaaaggaggcaacgaaaacaattttatccatacttacgattgccgacgaacgtgtcagtatacagtctgatgcatccgctctggcggtggtggcaatatctctaacactgttgaggagagccaagggcgaattc

SEQ ID NO: 5 - protein, 87 aa, Conus magus
MEGRRFAAVLILTLCMLASGAVAARPKDRPSYCNLPADSGSGTKPEQRIYYNSAREQCLTFTYNGKGGNENNFIHTYDCRRTCQYTV

SEQ ID NO: 6 - protein, 64 aa, Conus magus
  MISC_FEATURE (3)..(22): Xaa at position 3, 7, 13 and 22 may be Pro or Hyp
ARXKDRXSXCNLXADSGSGTKXXQRIXXNSARXQCLTFTXNGKGGNXNNFIHTXDCRRTCQXTV

SEQ ID NO: 7 - DNA, 576 bp, Conus magus
  CDS (264)..(512)
gaattcgcccttggcgatgaatgaacactgcgtttgctggctttgacgaaaaccagtccacgtcagcagagggagagagagagagagaccttaggagctgtgtattctccagtcatctgacggccccatccacgtcagtgctgtttcttctatcagccagtccatatcaacgctctaccgtctgtcagccagtccacatcagcgctctaccatctgtcagcctgtttacgtcagcactgtcatctgactgtccaggttaaaaaatggagggacgtcgttttgctgctgttctgatcctgaccatctgtatgcttgcacctgggactggtcgaccgagtgtatgcaataaaccagcggacaaggggccgtgcacagggtctgagaagagattctacttcagtacctatcataatgagtgtagaacgttcaaatacggaggatgtgaaggcaacggcaacaattttatccatgtgtacaattgtcgaagaacgtgtgtgtatccacctggctgaaataactgcgctgactgttgtggcgacatccctaacactgttgaggagagccaagggcgaattc

SEQ ID NO: 8 - protein, 82 aa, Conus magus
MEGRRFAAVLILTICMLAPGTGRPSVCNKPADKGPCTGSEKRFYFSTYHNECRTFKYGGCEGNGNNFIHVYNCRRTCVYPPG

SEQ ID NO: 9 - protein, 59 aa, Conus magus
  MISC_FEATURE (2)..(59): Xaa at positions 2, 8, 13, 58 and 59 may be Pro or Hyp
RXSVCNKXADKGXCTGSXKRFXFSTXHNXCRTFKXGGCXGNGNNFIHVXNCRRTCVXXX

SEQ ID NO: 10 - DNA, 597 bp, Conus magus
  CDS (270)..(533)
gaattcgcccttgctgatggcgatgaatgaacactgcgtttgctggctttgatgaaaaccagtccacgtcagcagagggagagagagagagagaccttaggagctgtgtattctccagtcatctgacggccccatccacgtcagtgctgtttcttctatcagccagtccatatcaacgctctaccgtctgtcagccagtccacatcagcgctctaccatctgtcagcctgtttacgtcagcactgtcatctgactgtccaggttaaaaaatggagggacgtcgttttgctgctgttctgatcctgaccctctgtatgcttgcatctggggctgtcgctgcgaaacctaagagtcggccgagttattgcaatctaccagcggacagtgggtcgggcacaaagtctgagaagagaatttactacaatagcgctagaaaacagtgtttaaggttcacttaccatggagaaggaggcaacgcaaacaattttctccatactttcgattgccaacatacgtgtctgtatacagtctgatgcatcccctctggcggtggtggcaatatctctaacactgttgaggagagccaagggcgaattc

SEQ ID NO: 11 - protein, 87 aa, Conus magus
MEGRRFAAVLILTLCMLASGAVAAKPKSRPSYCNLPADSGSGTKSEKRIYYNSARKQCLRFTYHGEGGNANNFLHTFDCQHTCLYTV

SEQ ID NO: 12 - protein, 64 aa, Conus magus
  MISC_FEATURE (3)..(13): Xaa at positions 3, 7 and 13 may be Pro or Hyp
AKXKSRXSXCNLXADSGSGTKSXKRIXXNSARKQCLRFTXHGXGGNANNFLHTFDCQHTCLXTV

SEQ ID NO: 13 - DNA, 361 bp, Conus magus
  CDS (13)..(297)
gaattcgcccttatggagggacgtcgttttgctgctgttctgatcctgaccatctgtatgcttgcacctgggactggtactttgatacctcggggtcgaccgagtgaatgcaatctacgaaaggacagtgggtcgtgcacaaggtctgagcagagcacaaagtctgagcagagatattacttcagtacatattataatgagtgtagaatgttcacttacagtggatgtggaggcaaccgcaacaattttatccatatgtacgattgtcgacgaaagtgtgtgtatacagttggctgaaataactgcgctgactgttgtggcgacatccctaacactgttgaggagagccaagggcgaattc

SEQ ID NO: 14 - protein, 94 aa, Conus magus
MEGRRFAAVLILTICMLAPGTGTLIPRGRPSECNLRKDSGSCTRSEQSTKSEQRYYFSTYYNECRMFTYSGCGGNRNNFIHMYDCRRKCVYTVG

SEQ ID NO: 15 - protein, 71 aa, Conus magus
  MISC_FEATURE (4)..(8): Xaa at positions 4 and 8 may be Pro or Hyp
TLIXRGRXSXCNLRKDSGSCTRSXQSTKSXQRXXFSTXXNXCRMFTXSGCGGNRNNFIHMXDCRRKCVXTV

SEQ ID NO: 16 - DNA, 340 bp, Conus magus
  CDS (13)..(276)
gaattcgcccttatggagggacgtcgttttgctgctgttctgatcctgaccctctgtatgcttgcatctggggctgtcgctgcgaaacctaagagtcggccgagttattgcaatctaccagcggagagggggccgtgcgaagggtcggagaagagatattatttcgatagctatcgtattcagtgtagattattcaaatacggaggatgtgaaggcaaccgcaacaattttatccgtgtgtacgattgtcgaaaaaagtgtttgcatgcaggctgatgcatccgctctggcggtggtggcgatatctctaacactgttgaggagagccaagggcgaattc

SEQ ID NO: 17 - protein, 87 aa, Conus magus
MEGRRFAAVLILTLCMLASGAVAAKPKSRPSYCNLPAERGPCEGSEKRYYFDSYRIQCRLFKYGGCEGNRNNFIRVYDCRKKCLHAG

SEQ ID NO: 18 - protein, 63 aa, Conus magus
  MISC_FEATURE (3)..(18): Xaa at positions 3, 7, 13 and 18 may be Pro or Hyp
AKXKSRXSXCNLXAXRGXCXGSXKRXXFDSXRIQCRLFKXGGCXGNRNNFIRVXDCRKKCLHA

SEQ ID NO: 19 - DNA, 346 bp, Conus magus
  CDS (13)..(282)
gaattcgcccttatggagggacgtcgttttgctgctgctgttctgatcctgaccctctgtatgcttgcacctggggctgtcgcttcgagacgtaaggctcgaccgagtgtatgcaatctaccaccggaaagggggccgtgcgcagggtctgagcggagagtttacttcgatagcacttctaaacagtgtaaaatgttcacttacggaggatgtggaggcaacggcaacaattttaaacgtgctcacgattgtttaaaaatgtgtgtgtattcagttggctgaaacaactgcgctgagtgttgtggcgacatccctaacactgttgaggagagccaagggcgaattc

SEQ ID NO: 20 - protein, 89 aa, Conus magus
MEGRRFAAAVLILTLCMLAPGAVASRRKARPSVCNLPPERGPCAGSERRVYFDSTSKQCKMFTYGGCGGNGNNFKRAHDCLKMCVYSVG

SEQ ID NO: 21 - protein, 64 aa, Conus magus
  MISC_FEATURE (7)..(18): Xaa at positions 7, 13, 14 and 18 may be Pro or Hyp
SRRKARXSVCNLXXXRGXCAGSXRRVXFDSTSKQCKMFTXGGCGGNGNNFKRAHDCLKMCVXSV

SEQ ID NO: 22 - DNA, 340 bp, Conus magus
  CDS (13)..(276)
gaattcgcccttatggagggacgtcgttttgctgctgttctgatcctgaccctctgtatgcttgcatctggggctgtcgctgcgaaacctaagagtcggccgagttattgcaatctaccagcggacagtgggtcgggcacaaagtctgagaagagaatttactacaatagcgctagaaaacagtgtttaaggttcacttaccatggagaaggaggcaacgcaaacaattttctccatactttcgattgccaacatacgtgtctgtatacagtctgatgcatcccctctggcggtggtggcaatatctctaacactgttgaggagagccaagggcgaattc

SEQ ID NO: 23 - protein, 87 aa, Conus magus; identical to SEQ ID NO: 11

SEQ ID NO: 24 - protein, 64 aa, Conus magus; identical to SEQ ID NO: 12
  MISC_FEATURE (3)..(13): Xaa at positions 3, 7 and 13 may be Pro or Hyp

SEQ ID NO: 25 - DNA, 340 bp, Conus striatus
  CDS (13)..(276)
gaattcgcccttatggagggacgtcgttttgctgctgttctgatcctgaccatctgtatgcttgcacctgggactggtactttgctacctcgggatcgaccgagtctatgcgatctaccagcggacagtgggtcgggcacaaaggctgagaagagaatttactacaatagcgctagaaaacagtgtttaaggttcgattacacaggacaaggaggcaacgaaaacaattttcgccgtacttacgattgccaacgaacgtgtctgtatacaggctgatgcatccgctctggtggtggtggcaatatctctaacactgttgaggagagccaagggcgaattc

SEQ ID NO: 26 - protein, 87 aa, Conus striatus
MEGRRFAAVLILTICMLAPGTGTLLPRDRPSLCDLPADSGSGTKAEKRIYYNSARKQCLRFDYTGQGGNENNFRRTYDCQRTCLYTG

SEQ ID NO: 27 - protein, 64 aa, Conus striatus
  MISC_FEATURE (4)..(14): Xaa at positions 4, 8 and 14 may be Pro or Hyp
TLLXRDRXSLCDLXADSGSGTKAXKRIXXNSARKQCLRFDXTGQGGNXNNFRRTXDCQRTCLXT

SEQ ID NO: 28 - DNA, 343 bp, Conus striatus
  CDS (13)..(279)
gaattcgcccttatggagggacgtcgttttgctgctgttctgatcctgaccctctgtatgcttgcatctggggctgtcgctgcgagacctaaggatcgaccgagttattgcaatctaccagcggacagtgggtcgggcacaaagcctgagcagagaatttactacaatagcgctaaaaaacagtgtgtaacgttcacttacaatggaaaaggaggcaacggcaacaattttagccgtactaacgattgccgacaaacgtgtcagtatccagttggctgaaataactgcgctgactggtgtggcgacatccctaacactgttgaggagagccaagggcgaattc

SEQ ID NO: 29 - protein, 88 aa, Conus striatus
MEGRRFAAVLILTLCMLASGAVAARPKDRPSYCNLPADSGSGTKPEQRIYYNSAKKQCVTFTYNGKGGNGNNFSRTNDCRQTCQYPVG

SEQ ID NO: 30 - protein, 64 aa, Conus striatus
  MISC_FEATURE (3)..(63): Xaa at positions 3, 7, 13, 22 and 63 may be Pro or Hyp
ARXKDRXSXCNLXADSGSGTKXXQRIXXNSAKKQCVTFTXNGKGGNGNNFSRTNDCRQTCQXXV

SEQ ID NO: 31 - DNA, 343 bp, Conus striatus
  CDS (13)..(279)
gaattcgcccttatggagggacgtcgttttgctgctgttctgatcctgaccctctgtatgcttgcatctcgggctgtcgctgcgagacctaaggatcgaccgagttattgcaatctaccagcggacagtgggtcgggcacaaagcctgagcagagaatttactacaatagcgctaaaaaacagtgtgtaacgttcacttacagtggaaaaggaggcaacggcaacaattttagccgtactaacgattgccgacaaacgtgtcagtatccagctggctgaaataactgcgctgactgttgtggcgacatccctaacactgttgaggagagccaagggcgaattc

SEQ ID NO: 32 - protein, 88 aa, Conus striatus
MEGRRFAAVLILTLCMLASRAVAARPKDRPSYCNLPADSGSGTKPEQRIYYNSAKKQCVTFTYSGKGGNGNNFSRTNDCRQTCQYPAG

SEQ ID NO: 33 - protein, 64 aa, Conus striatus
  MISC_FEATURE (3)..(63): Xaa at positions 3, 7, 13, 22 and 63 may be Pro or Hyp
ARXKDRXSXCNLXADSGSGTKXXQRIXXNSAKKQCVTFTXSGKGGNGNNFSRTNDCRQTCQXXA

SEQ ID NO: 34 - DNA, 340 bp, Conus striatus
  CDS (13)..(276)
gaattcgcccttatggagggacgtcgttttgctgctgttctgatcctgaccctctgtatgcttgcatctggggctgtcgctgcgaaacctaagagtcggccgagttattgcaatctaccagcggacagtgggtcgggcacaaagtctgagaagagaatttactacaatagcgctagaaaacagtgtttagggttcacttaccatggagaaggaggcaacgcaaacaactttctccatactttcgattgccaacatacgtgtctgtatacagtctgatgcatccgctctggcggtggtggcaatatctctaacactgttgaggagagccaagggcgaattc

SEQ ID NO: 35 - protein, 87 aa, Conus striatus
MEGRRFAAVLILTLCMLASGAVAAKPKSRPSYCNLPADSGSGTKSEKRIYYNSARKQCLGFTYHGEGGNANNFLHTFDCQHTCLYTV

SEQ ID NO: 36 - protein, 64 aa, Conus striatus
  MISC_FEATURE (3)..(13): Xaa at positions 3, 7 and 13 may be Pro or Hyp
AKXKSRXSXCNLXADSGSGTKSXKRIXXNSARKQCLGFTXHGXGGNANNFLHTFDCQHTCLXTV

SEQ ID NO: 37 - DNA, 349 bp, Conus consors
  CDS (13)..(285)
  misc_feature (227)..(227): n is a, c, g, or t
gaattcgcccttatggagggacgtcgttttgctgctgttctgatcctgaccctctgtatgcttgcatctggggctgtcgctgcgaaacctaagagtcggccgagttattgcaatctaccagcggacagtgggtcgggcacaaagtctgagaagagaatttactacaatagcgctagaaaacagtgtttaaggttcacttaccatggagaaagggaagggccaaacgncaaaaacaattttctccatactttcgattgccaacatacgtgtctgtatacagtctgatgcatccgctctggcggtggtggcaatatctctaacactgttgaggagagccaagggcgaattc

SEQ ID NO: 38 - protein, 90 aa, Conus consors
  misc_feature (72)..(72): The ′Xaa′ at location 72 stands for Asp, Gly, Ala, or Val.
MEGRRFAAVLILTLCMLASGAVAAKPKSRPSYCNLPADSGSGTKSEKRIYYNSARKQCLRFTYHGEREGPNXKNNFLHTFDCQHTCLYTV

SEQ ID NO: 39 - protein, 67 aa, Conus consors
  MISC_FEATURE (3)..(47): Xaa at positions 3, 7, 13 and 47 may be Pro or Hyp
AKXKSRXSXCNLXADSGSGTKSXKRIXXNSARKQCLRFTXHGXRXGXNXKNNFLHTFDCQHTCLXTV

SEQ ID NO: 40 - DNA, 340 bp, Conus consors
  CDS (13)..(276)
gaattcgcccttatggagggacgtcgttttgctgctgttctgatcctgaccctctgtatgcttgcatctggggctgtcgctgcgaaacctaagagtcggccgagttattgcaatctaccagcggacagtgggtcgggcacaaagtctgagaagagaatttactacaatagcgctagaaaacagtgtttaaggttcacttaccatggagaaggaggcaacgcaaacaattttctccatactttcgattgccaacatacgtgtctgtatacagtctgatgcatccgctctggcggtggtggcaatatctctaacactgttgaggagagccaagggcgaattc

SEQ ID NO: 41 - protein, 87 aa, Conus consors; identical to SEQ ID NO: 11

SEQ ID NO: 42 - protein, 64 aa, Conus consors; identical to SEQ ID NO: 12
  MISC_FEATURE (3)..(13): Xaa at positions 3, 7 and 13 may be Pro or Hyp

SEQ ID NO: 43 - DNA, 340 bp, Conus consors
  CDS (13)..(276)
gaattcgcccttatggagggacgtcgttttgctgctgttctgatcctgaccctctgtatgcttgcatctggggctgtcgctgcgagacctaaggatcgaccgagttattgcaatctaccagcggacagtgggtcgggcacaaagcctgagcagagaatttactacaatagcgctcgaaaacagtgtttaacgttcacttacaatggaaaaggaggcaacgaaaacaattttatccatacttacgattgccgacgaacgtgtcagtatacagtctgatgcatccgctctggcggtggtggcaatatctctaacactgttgaggagagccaagggcgaattc

SEQ ID NO: 44 - protein, 87 aa, Conus consors
MEGRRFAAVLILTLCMLASGAVAARPKDRPSYCNLPADSGSGTKPEQRIYYNSARKQCLTFTYNGKGGNENNFIHTYDCRRTCQYTV

SEQ ID NO: 45 - protein, 64 aa, Conus consors
  MISC_FEATURE (3)..(22): Xaa at positions 3, 7, 13 and 22 may be Pro or Hyp
ARXKDRXSXCNLXADSGSGTKXXQRIXXNSARKQCLTFTXNGKGGNXNNFIHTXDCRRTCQXTV

SEQ ID NO: 46 - DNA, 361 bp, Conus catus
  CDS (13)..(297)
gaattcgcccttatggagggacgtcgttttgctgctgttctgatcctgaccacctgtatgcttgcacctgggactggtactttgatacctcggggtcgaccgagttattgcaatctacgaaaggacagtgggtcgtgcacaaggtctgagcagagcacagagtctgagtacagatattacttcagtacacattataatgagtgtagaatgttcacttacaaaggatgtggaggcaaccgcaacaattttatccatatgcacgattgtcgacaaaagtgtgtgtatacagttggctgaaataattgcgctgactgttgtggcgacatccctaacactgttgaggagatccaagggcgaattc

SEQ ID NO: 47 - protein, 94 aa, Conus catus
MEGRRFAAVLILTTCMLAPGTGTLIPRGRPSYCNLRKDSGSCTRSEQSTESEYRYYFSTHYNECRMFTYKGCGGNRNNFIHMHDCRQKCVYTVG

SEQ ID NO: 48 - protein, 71 aa, Conus catus
  MISC_FEATURE (4)..(8): Xaa at positions 4 and 8 may be Pro or Hyp
TLIXRGRXSXCNLRKDSGSCTRSXQSTXSXXRXXFSTHXNXCRMFTXKGCGGNRNNFIHMHDCRQKCVXTV

SEQ ID NO: 49 - DNA, 346 bp, Conus catus
  CDS (13)..(282)
gaattcgcccttatggagggacgtcgttttgctgctgttctgatcctgaccatctgtatgcttgcacctgggactggtactttgatacctcgggatcgaccgagttattgcaatctaccagcggacagtgggtcgggcacaaatcgtgagcagagaatttactacaatagcgctagaaaacagtgtttaacgttcccttacaaaggaaaaggaggcaacgcaaacaattttagccgtactaacgattgccgacgaacgtgtcagtatccagcagctggctgaaataattgcgctgactgttgtggcgacatccctaacactgttgaggagagccaagggcgaattc

SEQ ID NO: 50 - protein, 89 aa, Conus catus
MEGRRFAAVLILTICMLAPGTGTLIPRDRPSYCNLPADSGSGTNREQRIYYNSARKQCLTFPYKGKGGNANNFSRTNDCRRTCQYPAAG

SEQ ID NO: 51 - protein, 66 aa, Conus catus
  MISC_FEATURE (4)..(64): Xaa at positions 4, 8, 14, 40 and 64 may be Pro or Hyp
TLIXRDRXSXCNLXADSGSGTNRXQRIXXNSARKQCLTFXXKGKGGNANNFSRTNDCRRTCQXXAA

SEQ ID NO: 52 - DNA, 527 bp, Conus betulinus
  CDS (13)..(468)
gaattcgcccttatggagggacgtcgttttgctgctgttctgatcctgaccatctgtctgctggcacttggggacggcgcttccagacgtcagggtccgccaacaaataagtgttctcgtgatgttgccctaagtaagtgttaccaacccagggatccggggccgtgtaatgctttctttgagaagtattacttcaacaggtatatcaacacttgcgaaaaattcacttacggaggatgtcaaggcaacgacaacagatttaacaccgtggaggactgcctaggatgttgtctactgagtgtatgcagacaaccagcggaaacgggaccgtgcagagggtatttcccgaaatattacttcgatatggattcttacgagtgcaaacagttcatttacggaggatgcggtggcaacgacaacaattttggcactgtcgaagagtgtcaaggcagatgtgggccgaaataactgcgctgattgttgtggcgacatccctaacactgttgaggagagccaagggcgaattc

SEQ ID NO: 53 - protein, 151 aa, Conus betulinus
MEGRRFAAVLILTICLLALGDGASRRQGPPTNKCSRDVALSKCYQPRDPGPCNAFFEKYYFNRYINTCEKFTYGGCQGNDNRFNTVEDCLGCCLLSVCRQPAETGPCRGYFPKYYFDMDSYECKQFIYGGCGGNDNNFGTVEECQGRCGPK

SEQ ID NO: 54 - protein, 129 aa, Conus betulinus
  MISC_FEATURE (7)..(128): Xaa at positions 7, 8, 24, 27, 29, 79, 84, 90 and 128 may be Pro or Hyp
ASRRQGXXTNKCSRDVALSKCXQXRDXGXCNAFFXKXXFNRXINTCXKFTXGGCQGNDNRFNTVXDCLGCCLLSVCRQXAXTGXCRGXFXKXXFDMDSXXCKQFIXGGCGGNDNNFGTVXXCQGRCGXK

SEQ ID NO: 55 - DNA, 527 bp, Conus betulinus
  CDS (13)..(468)
gaattcgcccttatggagggacgtcgttttgctgctgttctgatcctgaccatctgtctgcttgctcttggggctggcgcttccagacgtcagggtccgccaacaaacaagtgttctcgtgatgttgccctaagtaagtgttaccaacccagggatccggggccgtgttatgctttgttggagaggtattacttcaacaggtatatcaacacttgcgaaaaattcacttacggaggatgtcaaggcaacgacaacagatttaacaccatggaggattgcctaggatgttgtcaactgagtgtatgcagacaaccagcggaaccgggactgtgcagagggtatttcctgagacattacttcgatatggattttaacgagtgcaaagagttcatttacggaggatgcggtggcaacgacaacaattttctcactgacgaagagtgtcaaggcagatgtgggccggaataactgcgctgattgttgtggcgacatccctaacactgttgaggagagccaagggcgaattc

SEQ ID NO: 56 - protein, 151 aa, Conus betulinus
MEGRRFAAVLILTICLLALGAGASRRQGPPTNKCSRDVALSKCYQPRDPGPCYALLERYYFNRYINTCEKFTYGGCQGNDNRFNTMEDCLGCCQLSVCRQPAEPGLCRGYFLRHYFDMDFNECKEFIYGGCGGNDNNFLTDEECQGRCGPE

SEQ ID NO: 57 - protein, 128 aa, Conus betulinus
  MISC_FEATURE (6)..(127): Xaa at positions 6, 7, 23, 26, 28, 78, 81 and 127 may be Pro or Hyp
SRRQGXXTNKCSRDVALSKCXQXRDXGXCXALLXRXXFNRXINTCXKFTXGGCQGNDNRFNTMXDCLGCCQLSVCRQXAXXGLCRGXFLRHXFDMDFNXCKXFIXGGCGGNDNNFLTDXXCQGRCGXX

SEQ ID NO: 58 - DNA, 323 bp, Conus betulinus
  CDS (13)..(264)
gaattcgcccttatggagggacgtcgttttgctgctgctctggtcctgaccatttgtctgcttgcacttggggctgacgcttccagacgtcaggatctgtgcagtctaccagcggaaaagggaccgtgcttagcatatatgccgagattttacttcgattgggattctaagttatgcaaagtgttcatttacggaggatgtcagggcaacgacaacaattttcacagttccacggactgttacgacacatgttggtttgaataactgcgctgattgttgtggcgacatccctaacactgttgaggagagccaagggcgaattc

SEQ ID NO: 59 - protein, 83 aa, Conus betulinus
MEGRRFAAALVLTICLLALGADASRRQDLCSLPAEKGPCLAYMPRFYFDWDSKLCKVFIYGGCQGNDNNFHSSTDCYDTCWFE

SEQ ID NO: 60 - protein, 60 aa, Conus betulinus
  MISC_FEATURE (10)..(21): Xaa at positions 10, 15 and 21 may be Pro or Hyp
SRRQDLCSLXAXKGXCLAXMXRFXFDXDSKLCKVFIXGGCQGNDNNFHSSTDCXDTCXFX

SEQ ID NO: 61 - DNA, 323 bp, Conus betulinus
  CDS (13)..(264)
gaattcgcccttatggagggacgtcgttttgctgctgttctgatcctgaccatctgtctgcttgcacttggggctggcgcttccagacgtcaggatctgtgcagtctaccagcggaaaagggaccgtgcttagcatatatgccgagattttacttcgattgggattctaagttatgcaaagtgttcatttacggaggatgtcagggcaacgacaacaattttcacagttcctcggactgttacgacacatgttggtttgaataactgcgctgattgttgtggcgacacccctaacactgttgaggagagccaagggcgaattc

SEQ ID NO: 62 - protein, 83 aa, Conus betulinus
MEGRRFAAVLILTICLLALGAGASRRQDLCSLPAEKGPCLAYMPRFYFDWDSKLCKVFIYGGCQGNDNNFHSSSDCYDTCWFE

SEQ ID NO: 63 - protein, 60 aa, Conus betulinus
  MISC_FEATURE (10)..(21): Xaa at positions 10, 15 and 21 may be Pro or Hyp
SRRQDLCSLXAXKGXCLAXMXRFXFDXDSKLCKVFIXGGCQGNDNNFHSSSDCXDTCXFX

SEQ ID NO: 64 - DNA, 527 bp, Conus betulinus
  CDS (13)..(468)
gaattcgcccttatggagggacgtcgttttgctgctgttctgatcctgaccatctgtctgctggcacttggggctggcgcttccaaacgtcagggtccgccaacaaacaagtgttctcgtgatgtcgccctaagtaagtgttaccaacccagggatccggggccgtgttatgcttcgtttcgcatgtattacttcaacaggtatatcaacacttgcgaaaaattcacttacggaggatgtcaaggcaacgacaacagatttaacaccgtggaggactgcctaggatgttgtcaactgagtgtatgcagacaaccagcggaaccgggactgtgcaaagggtatttcccgagacattacttcgatatggattcttacgagtgcaaaacgttcatttacggaggatgcggtggcaacgacaacaattttctcactgacggagagtgtcaaggcagatgtgggccggaataactgcgctgattgttgtggcgacatccctaacactgttgaggagagccaagggcgaattc

SEQ ID NO: 65 - protein, 151 aa, Conus betulinus
MEGRRFAAVLILTICLLALGAGASKRQGPPTNKCSRDVALSKCYQPRDPGPCYASFRMYYFNRYINTCEKFTYGGCQGNDNRFNTVEDCLGCCQLSVCRQPAEPGLCKGYFPRHYFDMDSYECKTFIYGGCGGNDNNFLTDGECQGRCGPE

SEQ ID NO: 66 - protein, 128 aa, Conus betulinus
  MISC_FEATURE (6)..(127): Xaa at positions 6, 7, 23, 26, 28, 78, 81, 89 and 127 may be Pro or Hyp
SKRQGXXTNKCSRDVALSKCXQXRDXGXCXASFRMXXFNRXINTCXKFTXGGCQGNDNRFNTVXDCLGCCQLSVCRQXAXXGLCKGXFXRHXFDMDSXXCKTFIXGGCGGNDNNFLTDGXCQGRCGXX

SEQ ID NO: 67 - DNA, 527 bp, Conus biliosus
  CDS (13)..(468)
gaattcgcccttatggagggacgtcgttttgctgctgttatgatcctcaccatctgcgtgcttgcacttagggctggcgctttcagacttcgtggttcgcgagctgaacagtgttctcgttctgaagccttagcaaagtgttaccttcccaaggatccggggccgtgtaaggcatggaagtccaggtattacttcaacagatacctcaacacgtgccaagagttcagttacggaggatgtcgtggcaacgccaacagatttgacaccatggaggactgccaagaatgttgtctactgagtgtatgcagacaaccagcggaaccggggctgtgcaaagcgtatatggagagatattactttgatatggattcttacgactgtaagccgttcatttacggaggatgtaacggcaacgacaacaattttctcacttacattgagtgttacggcagatgtgggccggaataactgcgctggttgttgtgccgacacccctaacactgttgaggagagccaagggcgaattc

SEQ ID NO: 68 - protein, 151 aa, Conus biliosus
MEGRRFAAVMILTICVLALRAGAFRLRGSRAEQCSRSEALAKCYLPKDPGPCKAWKSRYYFNRYLNTCQEFSYGGCRGNANRFDTMEDCQECCLLSVCRQPAEPGLCKAYMERYYFDMDSYDCKPFIYGGCNGNDNNFLTYIECYGRCGPE

SEQ ID NO: 69 - protein, 128 aa, Conus biliosus
  MISC_FEATURE (9)..(128): Xaa at positions 9, 15, 47, 64, 68, 80, 89, 120 and 128 may be Glu or gamma-carboxy-Glu
FRLRGSRAXQCSRSXALAKCXLXKDXGXCKAXKSRXXFNRXLNTCQXFSXGGCRGNANRFDTMXDCQXCCLLSVCRQXAXXGLCKAXMXRXXFDMDSXDCKXFIXGGCNGNDNNFLTXIXCXGRCGXX

SEQ ID NO: 70 - DNA, 527 bp, Conus biliosus
  CDS (13)..(468)
gaattcgcccttatggagggacgtcgttttgctgctgtactgatcctcaccatctgcgtgcttgcacttagggctggcgctttcagacttcgtggttcgcgagctgaagagtgttctcgttctgaagccttagcaaagtgttaccttcccaaggatccggggccgtgtaaggcatggaagtccaggtattacttcaacagatacctcaacacgtgccaagagttcagttacggaggatgtcgtggcaacgccaacagatttgacaccatggaggactgccaagaatgttgtctactgagtgtatgcagacaaccagcggaaccggggctgtgcaaagcgtatatggagagatattactttgatatggattcttacgactgtaagccgttcatttacggaggatgtaacggcaacgacaacaattttctcacttacattgagtgttacggcagatgtgggccggaataactgcgctggttgttgtgccgacacccctaacactgttgaggagagccaagggcgaattc

SEQ ID NO: 71 - protein, 151 aa, Conus biliosus
MEGRRFAAVLILTICVLALRAGAFRLRGSRAEECSRSEALAKCYLPKDPGPCKAWKSRYYFNRYLNTCQEFSYGGCRGNANRFDTMEDCQECCLLSVCRQPAEPGLCKAYMERYYFDMDSYDCKPFIYGGCNGNDNNFLTYIECYGRCGPE

SEQ ID NO: 72 - protein, 128 aa, Conus biliosus
  MISC_FEATURE (9)..(128): Xaa at positions 9, 10, 15, 47, 64, 68, 80, 89, 120 and 128 may be Glu or gamma-carboxy-Glu
FRLRGSRAXXCSRSXALAKCXLXKDXGXCKAXKSRXXFNRXLNTCQXFSXGGCRGNANRFDTMXDCQXCCLLSVCRQXAXXGLCKAXMXRXXFDMDSXDCKXFIXGGCNGNDNNFLTXIXCXGRCGXX

SEQ ID NO: 73 - DNA, 384 bp, Conus wittigi
  CDS (13)..(273)
gaattcgcccttatggagggacgtcgttttgctgctgttctgatcctgaccatctgtatgcttgcacttggggctggtgcttcgagacttcgggatcaactgaatagatgcaaacaaccaaaggtaacggggccgtgcaaaggggcgtttcggaggtattactttgatacagatgttcgcaactgtaaacagttcacttacggaggatgtagaggcaacgacaacaattttctcacttatgatgactgtaacgccacatgtggtccgaaataattgcgctgattgttgtggcgacatccctagcactgctgaggagagccacgctgtgtgctgattgttgtggcgacatccctaacactgttgaggagagccaagggcgaattc

SEQ ID NO: 74 - protein, 86 aa, Conus wittigi
MEGRRFAAVLILTICMLALGAGASRLRDQLNRCKQPKVTGPCKGAFRRYYFDTDVRNCKQFTYGGCRGNDNNFLTYDDCNATCGPK

SEQ ID NO: 75 - protein, 63 aa, Conus wittigi
  MISC_FEATURE (13)..(62): Xaa at positions 13, 18 and 62 may be Pro or Hyp
SRLRDQLNRCKQXKVTGXCKGAFRRXXFDTDVRNCKQFTXGGCRGNDNNFLTXDDCNATCGXK

SEQ ID NO: 76 - DNA, 1315 bp, Conus distans
  CDS (136)..(822)
  misc_feature (687)..(1238): n is a, c, g or t
gaattcggccattacggccgggatctacactccgcctgctgacacgtcatctactctctgtctccctgactgctgccttcagtcgaccctgccgtcatctcagcgcagacttggtaagaagtgaaaacctttatcatggagaaactgataaccctgcttcttgttgctactgtcctattgttgaccctggttcaagcaatagcagaaaacacctgcgaacagccaccggagccaggtaggtgtgagggatactatcgtaggtttttctacaactggaggtcgggccagtgcgagtcatttgtctatggaggttgtggtggcaacgacaacaatttccggaccaacgctgaatgtgaggctgcgtgctccagtaccaacacttatgctatatcagaaaacacctgcgaacagccagcggagcaaggtaggtgtacggagtacgttgtcaggtttttctacaactggaagtcggacaagtgcgagccatttgtctatggaggttgtggtggcaacagcaacagtttcctgaccatcactgaatgtgagactgcatgctccagtggaaacacttatgaagacgtatgccaactgccagctgatcgaggggtatgttcggcgcacttgcacatgttttactataactcctcatcaggacagtgccatcagtttatctacagaggttgtggnggtaacggcaacaaattttcgaccatgagggaatgtcaagctaagtgctccagaggcgtaaagtactcaaaggtcttgccaacaaaacaatggaaattttttagacgaaacgagccccgatatttgttggcatgagtaaaagaggaaattgancagcncggagaggtnccnnacggagtnccnggggtgcttggggattcaggacnccctgctgctgctgttgcatgcggctggaatccttggcgttntttgacagtttnccccagangaancctttgcttgacatcncgttttttccctttttntctttttgtagatcgtttttaacctgttggngttttcanttagaacagcgngttttcagatgtncagtatccangtntatgngatntctttgtctgccngtctgtctgtctgtctgtctgtntntcaatntgagattttagtcagaacaatatncntttaaacataaagttttcaanccaangtatttntagtttttcccttttnttttaccnctnttatttttccccntgatttnccnttaaacnttgtttttctaaaaaaaaaaaaaaaaaaaaaaaaaaaaaacatgtcggccgcctcggccctcgagaagctttctaga

SEQ ID NO: 77 - protein, 228 aa, Conus distans
MEKLITLLLVATVLLLTLVQAIAENTCEQPPEPGRCEGYYRRFFYNWRSGQCESFVYGGCGGNDNNFRTNAECEAACSSTNTYAISENTCEQPAEQGRCTEYVVRFFYNWKSDKCEPFVYGGCGGNSNSFLTITECETACSSGNTYEDVCQLPADRGVCSAHLHMFYYNSSSGQCHQFIYRGCGGNGNKFSTMRECQAKCSRGVKYSKVLPTKQWKFFRRNEPRYLLA

SEQ ID NO: 78 - protein, 205 aa, Conus distans
  MISC_FEATURE (1)..(199): Xaa at positions 1, 5, 9, 14, 30, 49, 51, 64, 68, 72, 78, 93, 112, 114, 124, 172, 199 may be Glu or gamma-carboxy-Glu
XNTCXQXXXXGRCXGXXRRFFXNXRSGQCXSFVXGGCGGNDNNFRTNAXCXAACSSTNTXAISXNTCXQXAXQGRCTXXVVRFFXNXKSDKCXXFVXGGCGGNSNSFLTITXCXTACSSGNTXXDVCQLXADRGVCSAHLHMFXXNSSSGQCHQFIXRGCGGNGNKFSTMRXCQAKCSRGVKXSKVLXTKQXKFFRRNXXRXLLA

SEQ ID NO: 79 - DNA, 579 bp, Conus distans
  CDS (156)..(401)
gaattcggccattacggccggggattcaagcgacgaggtaggtgaagtgaaggcgagacgtaggtgcatttctctactgagtgctgctgtgttctgccttgacctgcctcagcccggtcagaaatacagtgggctacaacacggaggttctggcgatgtcaaaactgggagtcgtgctgatcacctttctgcttctgtttcccctgggaactgttcagctgggtggagatgaccgctgcacactgcagttatcccacggcacctgtccaggctatttcccccgctggttctatgactcggccagtggccagtgccagagcttcatatacagcggatgtaaaggcaacgccaacaactttctgtctgaggaggaatgccgccaggcttgcgtcagccgttgacgtcaattataccacgttccttccttccgtcgtgatggagatttcaatcttgtcatcggtagaagccagtctcgtcgttctatgtgtatgacgtcaccgttttatggtgtgacgtcaccgttttatggatgtggcgtggcctgaaactgaaaaggtataaaaacatcagaaatctact

SEQ ID NO: 80 - protein, 81 aa, Conus distans
MSKLGVVLITFLLLFPLGTVQLGGDDRCTLQLSHGTCPGYFPRWFYDSASGQCQSFIYSGCKGNANNFLSEEECRQACVSR

SEQ ID NO: 81 - protein, 59 aa, Conus distans
  MISC_FEATURE (16)..(20): Xaa at positions 16 and 20 may be Pro or Hyp
GGDDRCTLQLSHGTCXGXFXRXFXDSASGQCQSFIXSGCKGNANNFLSXXXCRQACVSR

SEQ ID NO: 82 - DNA, 340 bp, Conus striatus; identical to SEQ ID NO: 25
  CDS (13)..(276)

SEQ ID NO: 83 - protein, 87 aa, Conus striatus; identical to SEQ ID NO: 26

SEQ ID NO: 84 - protein, 64 aa, Conus striatus; identical to SEQ ID NO: 27
  MISC_FEATURE (4)..(14): Xaa at positions 4, 8 and 14 may be Pro or Hyp

SEQ ID NO: 85 - DNA, 2298 bp, Conus magus
  CDS (264)..(512)
gaattcgcccttggcgatgaatgaacactgcgtttgctggctttgacgaaaaccagtccacgtcagcagagggagagagagagagagaccttaggagctgtgtattctccagtcatctgacggccccatccacgtcagtgctgtttcttctatcagccagtccatatcaacgctctaccgtctgtcagccagtccacatcagcgctctaccatctgtcagcctgtttacgtcagcactgtcatctgactgtccaggttaaaaaatggagggacgtcgttttgctgctgttctgatcctgaccatctgtatgcttgcacctgggactggtcgaccgagtgtatgcaataaaccagcggacaaggggccgtgcacagggtctgagaagagattctacttcagtacctatcataatgagtgtagaacgttcaaatacggaggatgtgaaggcaacggcaacaattttatccatgtgtacaattgtcgaagaacgtgtgtgtatccacctggctgaaataactgcgctgactgttgtggcgacatccctaacactgttgaggagagccaagctgtgggcttctacagatcctttctacagcgtctagtctgtgttctgtatctctgtggatccttttaatggtatctgttttgggtatctctatagatactttacagagggtctgttttgtgtttctgtacagatccttttcaaagtgtctgttttgtgtatctctacaggtccttttcaaagtgtctgttttgtgtatctctacagatccttttcgtagtgtctgttttgtgtatcgctacagatccttttcaaagtgttttgtgtatctctacagatccttttcaaattgttttgtgtatctctacagatccttttcaaagtgtctgtttgtgtacctctacagatacttcccatagagtctgttatagagtctataaatcctttctatagcatctctttttatgcgcctgtacatatccgtttcactgcatctattttgtgcatctctatagatcctttccatagtgtctgtactctgtattccaaaagattctttccgtggtgtctttcttgtgcaccactacgcatcctttccatagtatctgtcttgattatctccccacactctttactcggtgctttctccttatttcacccccttctcattctgtgttgattctccaatcctatctttctcctgcttccttttttccctccacttgtagtacttgttcgtcttctgagctcttttttattttttacttctcctgttcttaaaaaagggcattgaataaaattatgtcagatgaatcaatgtaaatgttatctacagacaagagagaaccttttttttttggtacgtcttccaaacgacacacatatgaatctgtggaaaaagttatttacagataagaagcgagtttctttttttaaacatatactccaaaatatgtacacgatcgccatgtgccctaccagtctgactgtcatcttttgatttttgatattaggatgcgttctttttttttttttttaactaaaactattctagccaaacatcttcaacactatctaaatgtcataaagaatgcatgaatcgtttaacactatttagagaataagattactttaggtatcaggctttatatgtatgatagttttgttattctatccattggcatcgtgcctgctgaaccagatatacacatcactttttttattagaaagtagtcgtcagaaatttggaaaaaaaagtcaaacggctattttgtatgttttccagaatttatggttgagacgcatttacattgttctgtttgcataatgtttgcttggcttcgccagtgtgtatttgtaatagcctggtgcggcttgaaaaccccaaggctgaagtgtcgccaaatctcgtggagcaaaatcaggaaggaggaggacagggcgaccttaaaactccatctactcagcactgtttggattgcctcatcatttcctttcaatacttttcgttttaaacgttggtgtctattggatttttcttcattattcaccacttaatctacggtggaggatctttgttcccgtctttacctctttgtctgctttgtacgtgcttctgttcttctttgaatcgaacagtaaatgtctgctttgtccatttaggggagtggtgtccccttgttttgaaatcttgtgaactggtttctgccaattaaacctattttctctgtagaagcgaaaaaaaaaaaaaaaaaaagtactctgcgttgttactcggcttaagggcgaattc

SEQ ID NO: 86 - protein, 82 aa, Conus magus; identical to SEQ ID NO: 8

SEQ ID NO: 87 - protein, 59 aa, Conus magus; identical to SEQ ID NO: 9
  MISC_FEATURE (2)..(59): Xaa at positions 2, 8, 13, 58 and 59 may be Pro or Hyp

SEQ ID NO: 88 - protein, 59 aa, Conus magus
  MISC_FEATURE (2)..(58): Xaa at positions 2, 8, 13 and 58 may be Pro or Hyp
RXSVCNKXADKGXCAGSXKRFXFSTXHNXCRTFKXGGCXGNGNNFIHVXNCRRTCVXXM

SEQ ID NO: 89 - protein, 60 aa, Conus striatus
  MISC_FEATURE (4)..(10): Xaa at positions 4 and 10 may be Pro or Hyp
KDRXSLCDLXADSGSGTKAXKRIXXNSARKQCLRFDXTGQGGNXNNFRRTXDCQRTCLXT

SEQ ID NO: 90 - protein, 52 aa, Conus striatus
  MISC_FEATURE (3)..(22): Xaa at positions 3, 7, 13 and 22 may be Pro or Hyp
GRXKDRXSXCNLXADSGSGTKXXQRIXXNSAKKQCVTFTXNGKGGNGNNFSR

SEQ ID NO: 91 - protein, 60 aa, Conus betulinus
SRRQDLCSLPAEKGPCLAYMPRFYFDWDSKLCKVFIYGGCQGNDNNFHSSTDCYDTCWFE

SEQ ID NO: 92 - protein, 60 aa, Conus betulinus
SRRQDLCSLPAEKGPCLAYMPRFYFDWDSKLCKVFIYGGCQGNDNNFHSSSDCYDTCWFE

SEQ ID NO: 93 - protein, 59 aa, Conus magus
RPSVCNKPADKGPCTGSEKRFYFSTYHNECRTFKYGGCEGNGNNFIHVYNCRRTCVYPP

SEQ ID NO: 94 - protein, 59 aa, Conus magus; identical to SEQ ID NO: 93

SEQ ID NO: 95 - protein, 62 aa, Conus magus
SRRKARPSVCNLPPERGPCAGSRVYFDSTSKQCKMFTYGGCGGNGNNFKRAHDCLKMCVYSV

SEQ ID NO: 96 - protein, 63 aa, Conus wittigi
SRLRDQLNRCKQPKVTGPCKGAFRRYYFDTDVRNCKQFTYGGCRGNDNNFLTYDDCNATCGPK

SEQ ID NO: 97 - protein, 59 aa, Conus distans
GGDDRCTLQLSHGTCPGYFPRWFYDSASGQCQSFIYSGCKGNANNFLSEEECRQACVSR

SEQ ID NO: 98 - protein, 63 aa, Conus magus
AKPKSRPSYCNLPAERGPCEGSEKRYYFDSYRIQCRLFKYGGCEGNRNNFIRVYDCRKKCLHA

SEQ ID NO: 99 - protein, 71 aa, Conus magus
TLIPRGRPSECNLRKDSGSCTRSEQSTKSEQRYYFSTYYNECRMFTYSGCGGNRNNFIHMYDCRRKCVYTV

SEQ ID NO: 100 - protein, 71 aa, Conus catus
TLIPRGRPSYCNLRKDSGSCTRSEQSTESEYRYYFSTHYNECRMFTYKGCGGNRNNFIHMHDCRQKCVYTV

SEQ ID NO: 101 - protein, 64 aa, Conus striatus
TLLPRDRPSLCDLPADSGSGTKAEKRIYYNSARKQCLRFDYTGQGGNENNFRRTYDCQRTCLYT

SEQ ID NO: 102 - protein, 64 aa, Conus striatus; identical to SEQ ID NO: 101

SEQ ID NO: 103 - protein, 66 aa, Conus catus
TLIPRDRPSYCNLPADSGSGTNREQRIYYNSARKQCLTFPYKGKGGNANNFSRTNDCRRTCQYPAA

SEQ ID NO: 104 - protein, 64 aa, Conus striatus
ARPKDRPSYCNLPADSGSGTKPEQRIYYNSAKKQCVTFTYNGKGGNGNNFSRTNDCRQTCQYPV

SEQ ID NO: 105 - protein, 64 aa, Conus consors
ARPKDRPSYCNLPADSGSGTKPEQRIYYNSARKQCLTFTYNGKGGNENNFIHTYDCRRTCQYTV

SEQ ID NO: 106 - protein, 64 aa, Conus magus
ARPKDRPSYCNLPADSGSGTKPEQRIYYNSAREQCLTFTYNGKGGNENNFIHTYDCRRTCQYTV

SEQ ID NO: 107 - protein, 64 aa, Conus striatus
ARPKDRPSYCNLPADSGSGTKPEQRIYYNSAKKQCVTFTYSGKGGNGNNFSRTNDCRQTCQYPA

SEQ ID NO: 108 - protein, 64 aa, Conus striatus
AKPKSRPSYCNLPADSGSGTKSEKRIYYNSARKQCLGFTYHGEGGNANNFLHTFDCQHTCLYTV

SEQ ID NO: 109 - protein, 64 aa, Conus magus
AKPKSRPSYCNLPADSGSGTKSEKRIYYNSARKQCLRFTYHGEGGNANNFLHTFDCQHTCLYTV

SEQ ID NO: 110 - protein, 64 aa, Conus consors; identical to SEQ ID NO: 109

SEQ ID NO: 111 - protein, 64 aa, Conus magus; identical to SEQ ID NO: 109

SEQ ID NO: 112 - protein, 67 aa, Conus consors
  MISC_FEATURE (49)..(49): Xaa may be Val, Ala, Asp or Gly
AKPKSRPSYCNLPADSGSGTKSEKRIYYNSARKQCLRFTYHGEREGPNXKNNFLHTFDCQHTCLYTV

SEQ ID NO: 113 - protein, 126 aa, Conus biliosus
FRLRGSRAEECSRSEAKCYLPKDPGPCKAWKSRYYFNRYLNTCQEFSYGGCRGNANRFDTMEDCQECCLLSVCRQPAEPGLCKAYMERYYFDMDSYDCKPFIYGGCNGNDNNFLTYIECYGRCGPE

SEQ ID NO: 114 - protein, 126 aa, Conus biliosus
FRLRGSRAEQCSRSEAKCYLPKDPGPCKAWKSRYYFNRYLNTCQEFSYGGCRGNANRFDTMEDCQECCLLSVCRQPAEPGLCKAYMERYYFDMDSYDCKPFIYGGCNGNDNNFLTYIECYGRCGPE

SEQ ID NO: 115 - protein, 126 aa, Conus betulinus
SKRQGPPTNKCSRDVSKCYQPRDPGPCYASFRMYYFNRYINTCEKFTYGGCQGNDNRFNTVEDCLGCCQLSVCRQPAEPGLCKGYFPRHYFDMDSYECKTFIYGGCGGNDNNFLTDGECQGRCGPE

SEQ ID NO: 116 - protein, 126 aa, Conus betulinus
SRRQGPPTNKCSRDVSKCYQPRDPGPCYALLERYYFNRYINTCEKFTYGGCQGNDNRFNTMEDCLGCCQLSVCRQPAEPGLCRGYFLRHYFDMDFNECKEFIYGGCGGNDNNFLTDEECQGRCGPE

SEQ ID NO: 117 - protein, 127 aa, Conus betulinus
ASRRQGPPTNKCSRDVSKCYQPRDPGPCNAFFEKYYFNRYINTCEKFTYGGCQGNDNRFNTVEDCLGCCLLSVCRQPAETGPCRGYFPKYYFDMDSYECKQFIYGGCGGNDNNFGTVEECQGRCGPK

SEQ ID NO: 118 - protein, 205 aa, Conus distans
ENTCEQPPEPGRCEGYYRRFFYNWRSGQCESFVYGGCGGNDNNFRTNAECEAACSSTNTYAISENTCEQPAEQGRCTEYVVRFFYNWKSDKCEPFVYGGCGGNSNSFLTITECETACSSGNTYEDVCQLPADRGVCSAHLHMFYYNSSSGQCHQFIYRGCGGNGNKFSTMRECQAKCSRGVKYSKVLPTKQWKFFRRNEPRYLLA

(19) United States
(12) Patent Application Publication
(10) Pub. No.: US 2006/0178303 A1
(43) Pub. Date: Aug. 10, 2006

(54) Potassium channel blockers

(75) Inventors: Baldomero M. Olivera, Salt Lake City, UT (US); Heinrich Terlau, Eckernfoerde (DE); Julita S. Imperial, Salt Lake City, UT (US); James E. Garrett, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignees: University of Utah Research Foundation, Salt Lake City, UT (US); Cognetix, Inc., Salt Lake City, UT (US); Maz-Planck-Gesellschaft zur Forderung der Wissenschaften e.V., Muchen (DE)

(21) Appl. No.: 11/222,216

(22) Filed: Sep. 9, 2005

Related U.S. Application Data
(60) Provisional application No. 60/608,153, filed on Sep. 9, 2004.

(51) Int. Cl. A61K 38/16; C07K 14/435

(52) U.S. Cl. 514/12; 530/324; 530/350

(57) ABSTRACT

The present invention is directed to conopeptides termed conkunitzins and their use for blocking the flow of potassium ions through voltage-gated potassium channels. In view of the Kunitz domain in the conkunitzins, they are also useful for inhibiting platelet aggregation and as protease inhibitors.

### CROSS-REFERENCE TO RELATED APPLICATION

The present application is related to and claims priority under 35 U.S. C. §119(e) to U.S. provisional patent application Ser. No. 60/608,153 filed on 9 Sep. 2004, incorporated herein by reference.

This invention was made with Government support under Grant No. GM-48677 awarded by the National Institutes of Health, Bethesda, Md. The United States Government has certain rights in the invention.

### BACKGROUND OF THE INVENTION

The present invention is directed to conopeptides termed conkunitzins and their use for blocking the flow of potassium ions through voltage-gated potassium channels. In view of the Kunitz domain in the conkunitzins, they are also useful for inhibiting platelet aggregation and as protease inhibitors.

The publications and other materials used herein to illuminate the background of the invention, and in particular, cases to provide additional details respecting the practice, are incorporated by reference, and for convenience are numerically referenced in the following text and respectively grouped in the appended bibliography.

Mollusks of the genus Conus produce a venom that enables them to carry out their unique predatory lifestyle. Prey are immobilized by the venom that is injected by means of a highly specialized venom apparatus, a disposable hollow tooth that functions both in the manner of a harpoon and a hypodermic needle.

Few interactions between organisms are more striking than those between a venomous animal and its envenomated victim. Venom may be used as a primary weapon to capture prey or as a defense mechanism. Many of these venoms contain molecules directed to receptors and ion channels of neuromuscular systems.

The predatory cone snails (Conus) have developed a unique biological strategy. Their venom contains relatively small peptides that are targeted to various neuromuscular receptors and may be equivalent in their pharmacological diversity to the alkaloids of plants or secondary metabolites of microorganisms. Many of these peptides are among the smallest nucleic acid-encoded translation products having defined conformations, and as such, they are somewhat unusual. Peptides in this size range normally equilibrate among many conformations. Proteins having a fixed conformation are generally much larger.

The cone snails that produce these toxic peptides, which are generally referred to as conotoxins or conotoxin peptides, are a large genus of venomous gastropods comprising approximately 500 species. All cone snail species are predators that inject venom to capture prey, and the spectrum of animals that the genus as a whole can envenomate is broad. A wide variety of hunting strategies are used, however, every Conusspecies uses fundamentally the same basic pattern of envenomation.

Several peptides isolated from Conusvenoms have been characterized. These include the α-, μ- and co-conotoxins which target nicotinic acetylcholine receptors, muscle sodium channels, and neuronal calcium channels, respectively (Olivera et al., 1985). Conopressins, which are vasopressin analogs, have also been identified (Cruz et al., 1987). In addition, peptides named conantokins have been isolated as well (Mena et al., 1990; Haack et al., 1990). These peptides have unusual age-dependent physiological effects: they induce a sleep-like state in mice younger than two weeks and hyperactive behavior in mice older than 3 weeks (Haack et al., 1990). The isolation, structure and activity of κ-conotoxins (now named κA conotoxins) are described in U.S. Pat. No. 5,633,347. Recently, peptides named contryphans containing D-tryptophan residues have been isolated from Conus radiatus (U.S. Pat. No. 6,077,934), and bromo-tryptophan conopeptides have been isolated from Conus imperialis and Conus radiatus (U.S. Pat. No. 5,889,147).

Potassium channels comprise a large and diverse group of proteins that, through maintenance of the cellular membrane potential, are fundamental in normal biological function. These channels are vital in controlling the resting membrane potential in excitable cells and can be broadly sub-divided into three classes: voltage-gated K⁺ channels, Ca²⁺ activated K⁺ channels and ATP-sensitive K⁺ channels. Many disorders are associated with abnormal flow of potassium ions through these channels. The identification of agents which would regulate the flow of potassium ions through each of these channel types would be useful in treating disorders associated with such abnormal flow.

It is desired to identify additional conotoxin peptides having activities of the above conopeptides, as well as conotoxin peptides having additional activities.

### SUMMARY OF THE INVENTION

The present invention is directed to conopeptides termed conkunitzins and their use for blocking the flow of potassium ions through voltage-gated potassium channels. The conkunitzins described herein are useful for treating various disorders as described in further detail herein. In view of the Kunitz domain in the conkunitzins, they are also useful for inhibiting platelet aggregation and as protease inhibitors.

In one embodiment, the present invention is directed to uses of the conkunitzins described herein for regulating the flow of potassium ions through K⁺ channels. Disorders which can be treated using these conopeptides include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction and disorders resulting from defects in neurotransmitter release (such as Eaton-Lambert syndrome). In addition, these conkunitzins can provide reversal of the actions of curare and other neuromuscular blocking drugs.

More specifically, the present invention is directed to conkunitzins, having the amino acid sequences set forth in Tables 1-5 below, as well as the corresponding propeptides and the nucleic acids that encode the propeptides that are set forth in Table 1.

The present invention is further directed to derivatives of the conkunitzins described herein or pharmaceutically acceptable salts of these peptides. Substitutions of one amino acid for another can be made at one or more additional sites within the described peptides, and may be made to modulate one or more of the properties of the peptides. Substitutions of this kind are preferably conservative, i.e., one amino acid is replaced with one of similar shape and charge. Conservative substitutions are well known in the art and include, for example: alanine to glycine, arginine to lysine, asparagine to glutamine or histidine, glycine to proline, leucine to valine or isoleucine, serine to threonine, phenylalanine to tyrosine, and the like.

Examples of derivatives include peptides in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated. The halogen may be iodo, chloro, fluoro or bromo; preferably iodo for halogen substituted-Tyr and bromo for halogen-substituted Trp. The Tyr residues may also be substituted with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂ up to and including n=8. The Leu residues may be substituted with Leu (D). The Glu residues may be substituted with Gla. The Gla residues may be substituted with Glu. The N-terminal Gln residues may be substituted with pyroGlu. The Met residues may be substituted with norleucine (Nle). The Cys residues may be in D or L configuration and may optionally be substituted with homocysteine (D or L).

The present invention is further directed to derivatives of the above peptides and peptide derivatives which are acylic permutations in which the cyclic permutants retain the native bridging pattern of native toxin. See Craik et al. (2001).

Examples of synthetic aromatic amino acids include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acids, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also their online catalog), incorporated herein by reference, by and available from RSP Amino Acid Analogues, Inc., Worcester, Mass. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference, and such as shown in the following schemes 1-3.

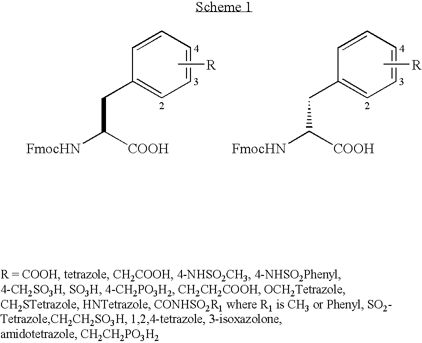

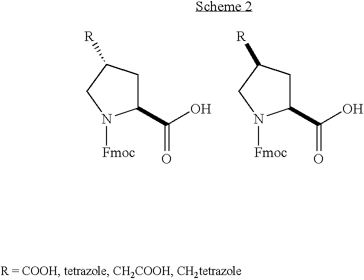

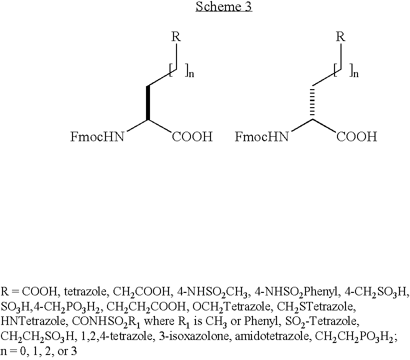

Optionally, in the conkunitzins of the present invention, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N—, S— or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The glycan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. patent applicantion Ser. No. 09/420,797 filed 19 Oct. 1999 and in Internatioinal Patent Application No. PCT/US99/24380 filed 19 Oct. 1999 (publication No. WO 00/23092), each incorporated herein by reference. A preferred glycan is Gal(β1→3)GalNAc(α1→).

Optionally, in the conkunitzins of the present invention, pairs of Cys residues may be replaced pairwise with isoteric lactam or ester-thioether replacements, such as Ser/(Glu or Asp), Lys/(Glu or Asp), Cys/(Glu or Asp) or Cys/Ala combinations. Sequential coupling by known methods (Bamay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues. In addition, individual Cys residues may be replaced with homoCys, seleno-Cys or penicillamine, so that disulfide bridges may be formed between Cys-homoCys or Cys-penicillamine, or homoCys-penicillamine and the like.

### DETAILED DESCRIPTION OF THE PREFERRED EMBODIMENTS

The present invention is directed conkunitzins and their uses as described above. Potassium channels comprise a large and diverse group of proteins that, through maintenance of the cellular membrane potential, are fundamental in normal biological function. The therapeutic applications for compounds that regulate the flow of potassium ions through K⁺ channels are far-reaching and include treatments of a wide range of disease and injury states. Disorders which can be treated using these conopeptides include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction and disorders resulting from defects in neurotransmitter release (such as Eaton-Lambert syndrome). In addition, these conkunitzins can provide reversal of the actions of curare and other neuromuscular blocking drugs.

The conkunitzins of the present invention are identified by isolation from Conus venom or by using recombinant DNA techniques by screening cDNA libraries of various Conus species using conventional techniques, such as the use of reverse-transcriptase polymerase chain reaction (RT-PCR) or the use of degenerate probes. Clones which hybridize to degenerate probes are analyzed to identify those which meet minimal size requirements, i.e., clones having approximately 400 nucleotides (for a propeptide), as determined using PCR primers which flank the cDNA cloning sites for the specific cDNA library being examined. These minimal-sized clones and the clones produced by RT-PCR are then sequenced. The sequences are then examined for the presence of a peptide having the characteristics noted above for conkunitzins. The biological activity of the peptides identified by this method is tested as described herein or conventionally in the art.

These peptides are sufficiently small to be chemically synthesized. General chemical syntheses for preparing the foregoing conopeptides are described hereinafter, along with specific chemical synthesis of conopeptides and indications of biological activities of these synthetic products. Various ones of these conopeptides can also be obtained by isolation and purification from specific Conusspecies using the techniques described in U.S. Pat. No. 4,447,356 (Olivera et al., 1984), U.S. Pat. No. 5,514,774 (Olivera et al., 1996) and U.S. Pat. No. 5,591,821 (Olivera et al., 1997), the disclosures of which are incorporated herein by reference.

Although the conopeptides of the present invention can be obtained by purification from cone snails, because the amounts of conopeptides obtainable from individual snails are very small, the desired substantially pure conopeptides are best practically obtained in commercially valuable amounts by chemical synthesis using solid-phase strategy. For example, the yield from a single cone snail may be about 10 micrograms or less of conopeptide. By “substantially pure” is meant that the peptide is present in the substantial absence of other biological molecules of the same type; it is preferably present in an amount of at least about 85% purity and preferably at least about 95% purity. Chemical synthesis of biologically active conopeptides depends of course upon correct determination of the amino acid sequence. Thus, the conopeptides of the present invention may be isolated, synthesized and/or substantially pure.

The conopeptides can also be produced by recombinant DNA techniques well known in the art. Such techniques are described by Sambrook et al. (1989). The peptides produced in this manner are isolated, reduced if necessary, and oxidized to form the correct disulfide bonds, if present in the final molecule.

One method of forming disulfide bonds in the conopeptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. It is also found that the linear peptide, or the oxidized product having more than one fraction, can sometimes be used for in vivo administration because the cross-linking and/or rearrangement which occurs in vivo has been found to create the biologically potent conopeptide molecule. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. No. 3,842,067 (1974) and U.S. Pat. No. 3,862,925 (1975). The synthesis of peptides containing γ-carboxyglutamic acid residues is exemplified by Rivier et al. (1987), Nishiuchi et al. (1993) and Zhou et al. (1996). Synthesis of conopeptides have been described in U.S. Pat. No. 4,447,356 (Olivera et al., 1984), U.S. Pat. No. 5,514,774 (Olivera et al., 1996) and U.S. Pat. No. 5,591,821 (Olivera et al., 1997).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or paramethylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH-MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in Horiki et al. (1978), using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder and Lubke (1965).

After removal of the a-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder and Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above. A suitable method for cyclization is the method described by Cartier et al. (1996).

Muteins, analogs or active fragments, of the foregoing conkunitzins are also contemplated here. See, e.g., Hammerland et al (1992). Derivative muteins, analogs or active fragments of the conkunitzins may be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 22, line 33); and U.S. Pat. No. 5,364,769 (see particularly col. 4, line 55 to col. 7, line 26), each incorporated herein by reference.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of the active ingredient will be admixed with a pharmaceutically acceptable carrier. The carrier may take a wide variety of forms depending on the form of preparation desired for administration, e.g., intravenous, oral or parenteral. For examples of delivery methods, see U.S. Pat. No. 5,844,077, incorporated herein by reference.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable for passage through the gastrointestinal tract, while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes into account the disorder to be treated, the condition of the individual patient, the site of delivery, the method of administration and other factors known to practitioners. Examples of techniques and protocols can be found in Remington's Pharmaceutical Sciences. Typically, the active agents of the present invention exhibit their effect at a dosage range of from about 0.001 mg/kg to about 250 mg/kg, preferably from about 0.01 mg/kg to about 100 mg/kg, of the active ingredient and more preferably, from about 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell-based delivery system in which a DNA sequence encoding an active agent is introduced into cells designed for implantation in the body of the patient, especially in the spinal cord region. Suitable delivery systems are described in U.S. Pat. No. 5,550,050 and in published PCT Applications No. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of developed sequences and the known genetic code.

### EXAMPLES

The present invention is described by reference to the following Examples, which are offered by way of illustration and are not intended to limit the invention in any manner. Standard techniques well known in the art or the techniques specifically described below were utilized.

Voltage-gated ion channels determine the membrane excitability of cells. Although many Conuspeptides that interact with voltage-gated Na⁺ and Ca⁺⁺ channels have been characterized, relatively few have been identified that interact with K⁺ channels. We describe novel Conuspeptides that interact with the Shaker K⁺ channel, the conkunitzins. The peptide was chemically synthesized.

### Example 1

### Experimental Procedures

Abbreviations: FMOC, N-(9-fluorenyl)methoxycarboxyl; HPLC; high performance liquid chromatography; i.c.v., intracerbrovascular; i.p., intraperitoneal; i.t, intrathecal; TFA, trifluoroacetic acid.

Synthesis: The peptides were synthesized on solid support using standard FMOC chemistry and a peptide synthesizer Symphony/Multiplex™ (Protein Technologies Inc.). Coupling time with HBTU and DIEA was 1.5 h. Peptide 32-60 cleavage/deprotection was accomplished with reagent K (82.5% TFA:5% phenol:5% water:5% thioanisole:2.5% EDT) for 4 hours at room temperature. Soluble crude peptide product was precipitated with cold MTBE, washed with MBTE and then dissolved in 25% aqueous acetonitrile, 1% TFA and lyophilized. Peptide was purified on a semi-preparative RP-HPLC column to 99% of purity.

For synthesis of the thioester peptide, peptide 1-31, the first amino acid was attached by mixing 4-sulfamylbutyryl AM resin (0.2 mmol), CH₂Cl₂ (5 mL), DIEA (342 μL, 2 mmol), and the Fmoc-Gln(Trt)-OH (1 mmol) (Backes and Ellman, 1999). The reaction mixture was stirred for 20 min, followed by cooling to −20° C. Then 420 mg of benzotriazole-1-yl-oxy-tris-pyrrolidino-phosphonium hexafluorophosphate (PyBOP) was added as a solid, and the reaction mixture was stirred for 12 h at −20° C., filtered and washed with CH₂Cl₂ (5×5 mL). The extent of incorporation was quantitated by the Fmoc release UV assay (Atherton and Sheppard, 1989). Coupling was repeated when loading <70%. The last Lys residue was introduced manually as a Boc-Lys(Boc)-OH derivative. Twenty mg of the resin-bound protein ( 1.0 mmol/g) was prepared for activation by initial addition 3 mL dried under solid Na tetrahydrofuran (THF) and after 15 min, 3 mL of 2 M TMS-CHN₂ (2 M solution in hexane) was added. After stirring on a rotary plated for 6 hours, the resin was washed with THF (5×5 mL) and DMF (5×5 mL) and used in the displacement reaction. The activated resin was swollen in DMF and drained. DMF (875 μL), ethyl-3-mercaptopropionate (130 μL, 1 mmol, 50 equiv) and sodium thiophenate (1.5 mg, 10 μmol, 0.5 equiv) was then added. The mixture was stirred on a rotary plated for 24 hours and the resin was filtered and washed with DMF (3×2 mL). The combined filtrate and washes were collected in a round-bottom flask and rotary evaporated, and then the oil was dried over KOH in vacuo for a minimum of 5 h. The side-chain-protecting groups on the crude peptide were removed with 2 mL of reagent B (88% TFA:5% phenol:2% triisopropylsilane:5% water) for 3 h at room temperature (Sole and Barany, 1992). The TFA solution was added dropwise to screw-cap centrifuge tubes containing MTBE with a TFA/MTBE ratio of 1/20. After centrifugation at 3200 g (30 min), the ether was removed and the peptide precipitate resuspended in MTBE. The process was repeated twice. The final precipitate was dissolved in 25% aqueous acetonitrile and lyophilized. Peptide was purified on a semi-preparative RP-HPLC column to 99% of purity.

To initiate native chemical ligation, peptide 1-31 (1.2 mg, 0.33 mol) and peptide 32-60 (1.75 mg, 0.5 μmol) were dissolved in 160 μL of 0.1 sodium phosphate buffer containing 6M GuHCL, pH 7.5 to which 4% thiopheno was added (1.6 μL) (Shin et al., 1999). The reaction was incubated for 10 h and monitored by analytical C18 reversed-phase HPLC separations in the linear gradient of acetonitrile from 10% to 60% buffer B in 45 minutes. The flow rate was 1 mL/min and the elution was monitored by UV detection at 220 nm. The final ligation product was purified on C18 reversed-phase HPLC column using semi-preparative column Vydac C-18 column (5 μm. 1 cm×25 cm) and a flow rated of 5 mL/min. The concentration of linear form of the peptide was determined spectrophotometrically using the molar absorbance coefficient at 274.5 nm, ε=1420 M⁻¹×cm⁻¹ (Litchfield and Wilcoxon, 1949).

Oxidative folding: Folding reactions were carried out in buffered solution (0.1 M Tris-HCL, 1 mM EDTA, pH 8.7) containing appropriate concentration of the linear peptide and a mixture of 0.5 mM reduced and 2.5 mM oxidized glutathione. The analytical folding reactions were initiated by adding 10 μL of the linear peptide, (resuspended in 0.01% TFA) to 40 μL folding mixture. The final peptide concentration was 20 μM. After appropriate time, the reaction was quenched by acidification with 5 μL of formic acid. The reaction mixtures was analyzed by analytical RP-HPLC (linear 40 min gradient from 21% to 25% buffer B) at 45° C. Peaks were collected and analyzed by MALDI-TOF on a Bruker Daltoncis OmniFLEX mass spectrometer using α-cyano-4-hydroxy cinnamic acid as matrix.

Disulfide mapping: Qualitative disulfide mapping of the peptide was performed with immobilized pepsin (Pierce Biotechnology). Immobilized pepsin, 100 μL was resuspended in 200 μL digest buffer (20 mM sodium acetate, pH 3.5). Peptide (10 nmoles) was dissolved in 200 μL digest buffer and 40 μL with pepsin was then added. The solution was incubated at 37° C. After 1 h, sample was stirred (2 min, 10,000 rpm) and 200 μL supernatant was applied to analytical RP-HPLC (linear 40 min gradient from 2% to 40% buffer B). Peaks were collected and analyzed by MALDI-TOF-MS.

Peptide corresponding to peak 5, about 8 nmoles, was dissolved in 160 μL digest buffer and 32 μL digest buffer with pepsin was added. The solution was incubated at 37° C. After 1 h, sample was stirred (2 min, 10,000 rpm) and 150 μL supernatant was applied to analytical RP-HPLC (linear 40 min gradient from 2% to 40% buffer B). Peaks were collected and analyzed by MALDI-TOF-MS.

Assessment of Proconvulsive Activity: Male CF-1 mice (26-35 g; Charles River Laboratories) were housed in a temperature controlled (23°±2° C.) room with a 12 hour light-dark cycle with free access to food and water. Conkunitzin ShK str-1 was administered to mice either by intraperitoneal injection (IP) or by freehand intracerebroventricular (ICV) injection (%:1 volume) using a 10:1 Hamilton syringe. Five doses (0.1, 0.3, 1, 2 and 3 nmol/mouse) of conkunitzin ShK str-1 with 3-5 mice per dose group were used for ED₅₀ determination. The percent of mice having seizures was calculated for each group, and the ED₅₀ and 95% confidence limits were calculated using probit analysis (Litchfield and Wilcoxon, 1949).

Electrophysiological methods: The Xenopus expression system was used for investigating the potential effects of the conkunitizins on voltage-gated Na⁺ and K⁺ channels. Oocytes from Xenopus laevis were prepared as described previously (Methfessel et al., 1986; Stühmer, 1992). Frogs were anaesthetized with 0.2% tricaine in ice water for surgery. Following cRNA injection, the oocytes were incubated 1-5 days to allow expression of the protein. Prior to the electrophysiological measurements, the vitelline membranes of the oocytes were removed mechanically with fine forceps. cRNAs encoding various cloned Na⁺ and K⁺ channels to be tested were prepared by standard techniques. Whole cell currents were recorded under two-electrode voltage clamp control using a Turbo-Tec amplifier (npi electronic, Tamm Germany). The intracellular electrodes were filled with 2 M KCl and had a resistance between 0.6 and 1 MΩ. Current records were low-pass filtered at 1 kHz (K⁺ channels) or 3 kHz (Na⁺ channels) (−3 dB) and sampled at 4 or 10 kHz, respectively. Leak and capacitive currents were corrected online by using a P/n method. The bath solution was normal frog Ringer's (NFR) containing (in mM): 115 NaCl, 2.5 KCl, 1.8 CaCl₂, 10 Hepes pH 7.2 (NaOH). Lyophilized conkunitzin was dissolved in NFR, diluted to the final concentration and added to the bath chamber. All electrophysiological experiments were performed at room temperature (19-22° C.).

Protein Isolation: Conkunitzin ShK str-1 and ShK str-2 were obtained by isolation and purification from Conusstriatus using the techniques described in U.S. Pat. No. 4,447,356 (Olivera et al., 1984), U.S. Pat. No. 5,514,774 (Olivera et al., 1996) and U.S. Pat. No. 5,591,821 (Olivera et al., 1997),

Molecular biology: The cDNA library from C. striatus venom ducts was prepared as previously described (Colledge et al., 1992; Jacobsen et al., 1998).

### Example 2

### Isolation of Conkunitzin ShK str-1, ShK str-2 and ShK magus

Two conkunitzins were isolated from Conus striatus and sequenced. One additional conkunitzin was isolated from Conus magus and sequenced. Conkunitzin ShK str-1 was found to have the following sequence:

(SEQ ID NO:1)

KDRPSLCDLPADSGSGTKAEKRIYYNSARKQCLRFDYTGQGGNENNFRRT
YDCQRTCLYT.

The Lys at position 1 may be replaced by an Arg.

Conkunitzin ShK str-2 was found to have the following partial sequence:

(SEQ ID NO:2)

GRPKDRPSYCNLPADSGSGTKPEQRIYYNSAKKQCVTFTYNGKGGNGNNF
SR.

Conkunitzin ShK magus was found to have the following sequence:

(SEQ ID NO:3)

RPSVCNKPADKGPCAGSEKRFYFSTYHNECRTFKYGGCEGNGNNFIHVYN
CRRTCVYPM\hat{ }

### Example 3

### Synthesis of Conkunitzin ShK str-1

To access synthetic conkunitzin by native chemical ligation strategy, two separate peptides were synthesized: Lys¹-Gln³¹-α-thiosester (peptide 1-31) and Cys³²-Thr⁶⁰ (peptide 32-60). Both peptides were prepared in a stepwise solid-phase method using Fmoc chemistry, purified by preparative RP-HPLC and characterized by MALDI mass spectrometry. Peptide Lys¹-Gln³¹-α-thioester was synthesized as described above. Ligation reaction was observed on RP-HPLC and all details are described above. After oxidative folding conkunitizin SHk str-1, the observed molecular mass decreased from 6934.43 Da (calculated mass of MH⁺=6934.66 Da, average) to 6930.41 Da (calculated mass of MH⁺=6930.66 Da, average), reflecting the loss of four protons because of the formation of two disulfide bonds.

### Example 4

### Folding of Conkunitzin ShK str-1

The 60-residue polypeptide chain of conkunitzin ShK str-1 contains four cysteines that form two disulfide bonds. The peptide was folded in buffered solution as described above. The yield of correct disulfide linkages was very high, with six intermediates observed on analytical RP-HPLC. Each intermediate was collected and analyzed by MALDI-TOF-MS. In the mass spectrum of intermediates I_{A}, I_{B}, I_{C} and I_{D}, a succession of peaks was observed at 6932.17, 6932.07, 6932.53 and 6932.31 Da (calculated mass of MH⁺=6932.66 Da, average), reflecting loss of two protons and indicating the presence of one disulfide bridge. In the mass spectrum of intermediates II_{A} and II_{B}, a succession of peaks was observed at 6930.80 and 6930.12 Da (calculated mass of MH⁺=6930.66 Da, average), reflecting loss of four protons and indicating the presence of two disulfide bridge.

### Example 5

### Disulfide Mapping of Conkunitzin ShK str-1

Enzymatic cleavage with pepsin in acidic conditions to reduce the potential for disulfide bond interchange was performed to generate individual disulfide-linkage peptides. Pepsin digestion of conkunitzin ShK str-1 was performed in two steps as described above. At the first step, conkunitzin ShK str-1 was cleaved into several linear fragments. One fragment was interpreted as being three linear peptides, KDRPSLCDLPADSGSGTKA (amino acids 1-19 of SEQ ID NO:1), NSARKQCLRF (amino acids 26-35 of SEQ ID NO:1) and RRTYDCQRTCL (amino acids 48-58 of SEQ ID NO:1), connected by two disulfide bonds and having the molecular mass of 4549.74 Da (calculated mass of MH⁺=4549.23 Da, monosiotopic). This peptide fragment was separated by analytical RP-HPLC and used in the second pepsin digest step. Mass spectrometric analyses of four additional peaks identified three ions corresponding to peptides linked by a single disulfide bond. The RP-HPLC peak 6 at 2161.46 Da (calculated mass of MH⁺=2161.06 Da, monoisotopic), represents peptide NSARKQCLRF(Cys2-Cys3)RRTYDCQ (amino acids 26-35 of SEQ ID NO:1 (Cys2-Cys3) amino acids 48-54 of SEQ ID NO:1). Peak 7 at 1960.31 Da (calculated mass of MH⁺=1599.99 Da, monoisotopic) represents peptide ARKQCLRF(Cys2-Cys3)RRTYDCQ (amino acids 28-35 of SEQ ID NO:1 (Cys2-Cys3) amino acids 48-54 of SEQ ID NO:1). Peak 8 at 2407.67 Da (calculated mass of MH+=2407.18 Da, monoisotopic) represents peptide KDRPSLCDLPADSGSGTKA(Cys1-Cys4)RTCL (amino acids 1-19 of SEQ ID NO:1 (Cys1-Cys4) amino acids 55-58 of SEQ ID NO:1). In summary, the linkage of the two disulfide bonds in conkunitzin ShK str-1 is identified as Cys1-Cys4 and Cys2-Cys3, by pepsin digestion followed by mass mapping.

### Example 6

### Cloning of Conkunitzin S1

Based upon the sequences of the mature peptides isolated from Conus magus and Conus striatus, degenerate primers were designed and used to isolate full-length cDNA clones for three conkunitzin peptides via 3′ and 5+ RACE techniques. The cDNA for each peptide encoded a precursor sequence containing a hydrophobic signal sequence N-terminal to the mature peptide. Primers were then designed to target the conserved 3′ and 5′ UTR regions of conkunitzins and were used to screen various Conusvenoms. The DNA sequences of the clones, the sequences of the encoded propeptides, and the sequences of the mature peptides are shown in Table 1. One of these, Conkunitzin S1, has been expressed in insect cell lines.

Most of the clones consist of a single kunitz domain. However, a few conkunitzins contain 2 or 3 kunitz domains in tandem. The conkunitzins can be grouped into four classes as shown in Tables 2-5. The groupings are based upon cysteine patterns (6 cysteines vs. 4 cysteines) and upon the number of tandem kunitz domains found in the peptide.

TABLE 1

Sequences of Mature Conkunitzins,
Propeptides and DNA Encoding Propeptides

| | |
|---|---|
| Name: C1679 | |
| Species: magus | |
| Isolated: No | |
| Cloned: Yes | |
| DNA Sequence: | (SEQ ID NO:4) |
| GAATTCGCCCTTGCTGATGGCGATGAATGAACACTG | |
| CGTTTGCTGGCTTTGATGAAAAGTCCACGTCAGCAG | |
| AGGGAGAGAGAGAGACCTTAGGAGCTGTGTATTCTC | |
| CAGTCATCTGACGGCCCTATCCACGTCAGTGCTGTT | |
| TCGTCTGTCAGCCAGTCCACATCACCGCTCAACCAT | |
| CTGTCAGCCTGTTTACGTCAGCACTGTCATCTGACT | |
| GTCCAGGTTAAAAAATGGAGGGACGTCGTTTTGCTG | |
| CTGTTCTGATCCTGACCCTCTGTATGCTTGCATCTG | |
| GGGCTGTCGCTGCGAGACCTAAGGATCGACCGAGTT | |
| ATTGCAATCTACCAGCGGACAGTGGGTCGGGCACAA | |
| AGCCTGAGCAGAGAATTTACTACAATAGCGCTCGAG | |
| AACAGTGTTTAACGTTCACTTACAATGGAAAAGGAG | |
| GCAACGAAAACAATTTTATCCATACTTACGATTGCC | |
| GACGAACGTGTCAGTATACAGTCTGATGCATCCGCT | |
| CTGGCGGTGGTGGCAATATCTCTAACACTGTTGAGG | |
| AGAGCCAAGGGCGAATTC | |
| Translation: | |
| MEGRRFAAVLILTLCMLASGAVAARPKDRPSYCNLP | (SEQ ID NO:5) |
| ASGSGTKPEQRIYYNSAREQCLTFTYNGKGGNENNF | |
| IHTYDCRRTCQYTV | |
| Toxin Sequence: | |
| Ala-Arg-Xaa3-Lys-Asp-Arg-Xaa3-Ser- | (SEQ ID NO:6) |
| Xaa5-Cys-Asn-Leu-Xaa3-Ala-Asp-Ser- | |
| Gly-Ser-Gly-Thr-Lys-Xaa3-Xaa1-Gln- | |
| Arg-Ile-Xaa5-Xaa5-Asn-Ser-Ala-Arg- | |
| Xaa1-Gln-Cys-Leu-Thr-Phe-Thr-Xaa5- | |
| Asn-Gly-Lys-Gly-Gly-Asn-Xaa1-Asn- | |
| Asn-Phe-Ile-His-Thr-Xaa5-Asp-Cys- | |
| Arg-Arg-Thr-Cys-Gln-Xaa5-Thr-Val-\hat{ } | |
| Name: C1687 | |
| Species: magus | |
| Isolated: No | |
| Cloned: Yes | |
| DNA Sequence: | |
| GAATTCGCCCTTGGCGATGAATGAACACTGCGTTTG | (SEQ ID NO:7) |
| CTGGCTTTGACGAAAACCAGTCCACGTCAGCAGAGG | |
| GAGAGAGAGAGAGAGACCTTAGGAGCTGTGTATTCT | |
| CCAGTCATCTGACGGCCCCATCCACGTCAGTGCTGT | |
| TTCTTCTATCAGCCAGTCCATATCAACGCTCTACCG | |
| TCTGTCAGCCAGTCCACATCAGCGCTCTACCATCTG | |
| TCAGCCTGTTTACGTCAGCACTGTCATCTGACTGTC | |
| CAGGTTAAAAAATGGAGGGACGTCGTTTTGCTGCTG | |
| TTCTGATCCTGACCATCTGTATGCTTGCACCTGGGA | |
| CTGGTCGACCGAGTGTATGCAATAAACCAGCGGACA | |
| AGGGGCCGTGCACAGGGTCTGAGAAGAGATTCTACT | |
| TCAGTACCTATCATAATGAGTGTAGAACGTTCAAAT | |
| ACGGAGGATGTGAAGGCAACGGCAACAATTTTATCC | |
| ATGTGTACAATTGTCGAAGAACGTGTGTGTATCCAC | |
| CTGGCTGAAATAACTGCGCTGACTGTTGTGGCGACA | |
| TCCCTAACACTGTTGAGGAGAGCCAAGGGCGAATTC | |
| Translation: | |
| MEGRRFAAVLILTICMLAPGTGRPSVCNKPADKGPC | (SEQ ID NO:8) |
| TGSEKRFYFSTYHNECRTFKYGGCEGNGNNFIHVYN | |
| CRRTCVYPPG | |
| Toxin Sequence: | |
| Arg-Xaa3-Ser-Val-Cys-Asn-Lys-Xaa3- | (SEQ ID NO:9) |
| Ala-Asp-Lys-Gly-Xaa3-Cys-Thr-Gly- | |
| Ser-Xaa1-Lys-Arg-Phe-Xaa5-Phe-Ser- | |
| Thr-Xaa5-His-Asn-Xaa1-Cys-Arg-Thr- | |
| Phe-Lys-Xaa5-Gly-Gly-Cys-Xaa1-Gly- | |
| Asn-Gly-Asn-Asn-Phe-Ile-His-Val- | |
| Xaa5-Asn-Cys-Arg-Arg-Thr-Cys-Val- | |
| Xaa5-Xaa3-Xaa3-# | |
| Name: C1689 | |
| Species: magus | |
| Isolated: No | |
| Cloned: Yes | |
| DNA Sequence: | |
| GAATTCGCCCTTGCTGATGGCGATGAATGAACACTG | (SEQ ID NO:10) |
| CGTTTGCTGGCTTTGATGAAAACCAGTCCACGTCAG | |
| CAGAGGGAGAGAGAGAGAGAGACCTTAGGAGCTGTG | |
| TATTCTCCAGTCATCTGACGGCCCCATCCACGTCAG | |
| TGCTGTTTCTTCTATCAGCCAGTCCATATCAACGCT | |
| CTACCGTCTGTCAGCCAGTCCACATCAGCGCTCTAC | |
| CATCTGTCAGCCTGTTTACGTCAGCACTGTCATCTG | |
| ACTGTCCAGGTTAAAAAATGGAGGGACGTCGTTTTG | |
| CTGCTGTTCTGATCCTGACCCTCTGTATGCTTGCAT | |
| CTGGGGCTGTCGCTGCGAAACCTAAGAGTCGGCCGA | |
| GTTATTGCAATCTACCAGCGGACAGTGGGTCGGGCA | |
| CAAAGTCTGAGAAGAGAATTTACTACAATAGCGCTA | |
| GAAAACAGTGTTTAAGGTTCACTTACCATGGAGAAG | |
| GAGGCAACGCAAACAATTTTCTCCATACTTTCGATT | |
| GCCAACATACGTGTCTGTATACAGTCTGATGCATCC | |
| CCTCTGGCGGTGGTGGCAATATCTCTAACACTGTTG | |
| AGGAGAGCCAAGGGCGAATTC | |
| Translation: | |
| MEGRRFAAVLILTLCMLASGAVAAKPKSRPSYCNLP | (SEQ ID NO:11) |
| ADSGSGTKSEKRIYYNSARKQCLRFTYHGEGGNANN | |
| FLHTFDCQHTCLYTV | |
| Toxin Sequence: | |
| Ala-Lys-Xaa3-Lys-Ser-Arg-Xaa3-Ser- | (SEQ ID NO:12) |
| Xaa5-Cys-Asn-Leu-Xaa3-Ala-Asp-Ser- | |
| Gly-Ser-Gly-Thr-Lys-Ser-Xaa1-Lys- | |
| Arg-Ile-Xaa5-Xaa5-Asn-Ser-Ala-Arg- | |
| Lys-Gln-Cys-Leu-Arg-Phe-Thr-Xaa5- | |
| His-Gly-Xaa1-Gly-Gly-Asn-Ala-Asn- | |
| Asn-Phe-Leu-His-Thr-Phe-Asp-Cys-Gln- | |
| His-Thr-Cys-Leu-Xaa5-Thr-Val\hat{ } | |
| Name: C1704 | |
| Species: magus | |
| Isolated: No | |
| Cloned: Yes | |
| DNA Sequence: | |
| GAATTCGCCCTTATGGAGGGACGTCGTTTTGCTGCT | (SEQ ID NO:13) |
| GTTCTGATCCTGACCATCTGTATGCTTGCACCTGGG | |
| ACTGGTACTTTGATACCTCGGGGTCGACCGAGTGAA | |
| TGCAATCTACGAAAGGACAGTGGGTCGTGCACAAGG | |
| TCTGAGCAGAGCACAAAGTCTGAGCAGAGATATTAC | |
| TTCAGTACATATTATAATGAGTGTAGAATGTTCACT | |
| TACAGTGGATGTGGAGGCAACCGCAACAATTTTATC | |
| CATATGTACGATTGTCGACGAAAGTGTGTGTATACA | |
| GTTGGCTGAAATAACTGCGCTGACTGTTGTGGCGAC | |
| ATCCCTAACACTGTTGAGGAGAGCCAAGGGCGAATT | |
| C | |
| Translation: | |
| MEGRRFAAVLILTICMLAPGTGTLIPRGRPSECNLR | (SEQ ID NO:14) |
| KDSGSCTRSEQSTKSEQRYYFSTYYNECRMFTYSGC | |
| GGNRNNFIHMYDCRRKCVYTVG* | |
| Toxin Sequence: | |
| Thr-Leu-Ile-Xaa3-Arg-Gly-Arg-Xaa3- | (SEQ ID NO:15) |
| Ser-Xaa1-Cys-Asn-Leu-Arg-Lys-Asp- | |
| Ser-Gly-Ser-Cys-Thr-Arg-Ser-Xaa1- | |
| Gln-Ser-Thr-Lys-Ser-Xaa1-Gln-Arg- | |
| Xaa5-Xaa5-Phe-Ser-Thr-Xaa5-Xaa5-Asn- | |
| Xaa1-Cys-Arg-Met-Phe-Thr-Xaa5-Ser- | |
| Gly-Cys-Gly-Gly-Asn-Arg-Asn-Asn-Phe- | |
| Ile-His-Met-Xaa5-Asp-Cys-Arg-Arg- | |
| Lys-Cys-Val-Xaa5-Thr-Val-# | |
| Name: C1705 | |
| Species: magus | |
| Isolated: No | |
| Cloned: Yes | |
| DNA Sequence: | |
| GAATTCGCCCTTATGGAGGGACGTCGTTTTGCTGCT | (SEQ ID NO:16) |
| GTTCTGATCCTGACCCTCTGTATGCTTGCATCTGGG | |
| GCTGTCGCTGCGAAACCTAAGAGTCGGCCGAGTTAT | |
| TGCAATCTACCAGCGGAGAGGGGGCCGTGCGAAGGG | |
| TCGGAGAAGAGATATTATTTCGATAGCTATCGTATT | |
| CAGTGTAGATTATTCAAATACGGAGGATGTGAAGGC | |
| AACCGCAACAATTTTATCCGTGTGTACGATTGTCGA | |
| AAAAAGTGTTTGCATGCAGGCTGATGCATCCGCTCT | |
| GGCGGTGGTGGCGATATCTCTAACACTGTTGAGGAG | |
| AGCCAAGGGCGAATTC | |
| Translation: | |
| MEGRRFAAVLILTLCMLASGAVAAKPKSRPSYCNLP | (SEQ ID NO:17) |
| AERGPCEGSEKRYYFDSYRIQCRLFKYGGCEGNRNN | |
| FIRVYDCRKKCLHAG | |
| Toxin Sequence: | |
| Ala-Lys-Xaa3-Lys-Ser-Arg-Xaa3-Ser- | (SEQ ID NO:18) |
| Xaa5-Cys-Asn-Leu-Xaa3-Ala-Xaa1-Arg- | |
| Gly-Xaa3-Cys-Xaa1-Gly-Ser-Xaa1-Lys- | |
| Arg-Xaa5-Xaa5-Phe-Asp-Ser-Xaa5-Arg- | |
| Ile-Gln-Cys-Arg-Leu-Phe-Lys-Xaa5- | |
| Gly-Gly-Cys-Xaa1-Gly-Asn-Arg-Asn- | |
| Asn-Phe-Ile-Arg-Val-Xaa5-Asp-Cys- | |
| Arg-Lys-Lys-Cys-Leu-His-Ala-# | |
| Name: C1708 | |
| Species: magus | |
| Isolated: No | |
| Cloned: Yes | |
| DNA Sequence: | |
| GAATTCGCCCTTATGGAGGGACGTCGTTTTGCTGCT | (SEQ ID NO:19) |
| GCTGTTCTGATCCTGACCCTCTGTATGCTTGCACCT | |
| GGGGCTGTCGCTTCGAGACGTAAGGCTCGACCGAGT | |
| GTATGCAATCTACCACCGGAAAGGGGGCCGTGCGCA | |
| GGGTCTGAGCGGAGAGTTTACTTCGATAGCACTTCT | |
| AAACAGTGTAAAATGTTCACTTACGGAGGATGTGGA | |
| GGCAACGGCAACAATTTTAAACGTGCTCACGATTGT | |
| TTAAAAATGTGTGTGTATTCAGTTGGCTGAAACAAC | |
| TGCGCTGAGTGTTGTGGCGACATCCCTAACACTGTT | |
| GAGGAGAGCCAAGGGCGAATTC | |
| Translation: | |
| MEGRRFAAAVLILTLCMLAPGAVASRRKARPSVCNL | (SEQ ID NO:20) |
| PPERGPCAGSERRVYFDSTSKQCKMFTYGGCGGNGN | |
| NFKRAHDCLKMCVYSVG | |
| Toxin Sequence: | |
| Ser-Arg-Arg-Lys-Ala-Arg-Xaa3-Ser- | (SEQ ID NO:21) |
| Val-Cys-Asn-Leu-Xaa3-Xaa3-Xaa1-Arg- | |
| Gly-Xaa3-Cys-Ala-Gly-Ser-Xaa1-Arg- | |
| Arg-Val-Xaa5-Phe-Asp-Ser-Thr-Ser- | |
| Lys-Gln-Cys-Lys-Met-Phe-Thr-Xaa5- | |
| Gly-Gly-Cys-Gly-Gly-Asn-Gly-Asn-Asn- | |
| Phe-Lys-Arg-Ala-His-Asp-Cys-Leu-Lys- | |
| Met-Cys-Val-Xaa5-Ser-Val-# | |
| Name: C1710 | |
| Species: magus | |
| Isolated: No | |
| Cloned: Yes | |
| DNA Sequence: | |
| GAATTCGCCCTTATGGAGGGACGTCGTTTTGCTGCT | (SEQ ID NO:22) |
| GTTCTGATCCTGACCCTCTGTATGCTTGCATCTGGG | |
| GCTGTCGCTGCGAAACCTAAGAGTCGGCCGAGTTAT | |
| TGCAATCTACCAGCGGACAGTGGGTCGGGCACAAAG | |
| TCTGAGAAGAGAATTTACTACAATAGCGCTAGAAAA | |
| CAGTGTTTAAGGTTCACTTACCATGGAGAAGGAGGC | |
| AACGCAAACAATTTTCTCCATACTTTCGATTGCCAA | |
| CATACGTGTCTGTATACAGTCTGATGCATCCCCTCT | |
| GGCGGTGGTGGCAATATCTCTAACACTGTTGAGGAG | |
| AGCCAAGGGCGAATTC | |
| Translation: | |
| MEGRRFAAVLILTLCMLASGAVAAKPKSRPSYCNLP | (SEQ ID NO:23) |
| ADSGSGTKSEKRIYYNSARKQCLRFTYHGEGGNANN | |
| FLHTFDCQHTCLYTV | |
| Toxin Sequence: | |
| Ala-Lys-Xaa3-Lys-Ser-Arg-Xaa3-Ser- | (SEQ ID NO:24) |
| Xaa5-Cys-Asn-Leu-Xaa3-Ala-Asp-Ser- | |
| Gly-Ser-Gly-Thr-Lys-Ser-Xaa1-Lys- | |
| Arg-Ile-Xaa5-Xaa5-Asn-Ser-Ala-Arg- | |
| Lys-Gln-Cys-Leu-Arg-Phe-Thr-Xaa5- | |
| His-Gly-Xaa1-Gly-Gly-Asn-Ala-Asn- | |
| Asn-Phe-Leu-His-Thr-Phe-Asp-Cys-Gln- | |
| His-Thr-Cys-Leu-Xaa5-Thr-Val-\hat{ } | |
| Name: C1716 | |
| Species: striatus | |
| Isolated: No | |
| Cloned: Yes | |
| DNA Sequence: | |
| GAATTCGCCCTTATGGAGGGACGTCGTTTTGCTGCT | (SEQ ID NO:25) |
| GTTCTGATCCTGACCATCTGTATGCTTGCACCTGGG | |
| ACTGGTACTTTGCTACCTCGGGATCGACCGAGTCTA | |
| TGCGATCTACCAGCGGACAGTGGGTCGGGCACAAAG | |
| GCTGAGAAGAGAATTTACTACAATAGCGCTAGAAAA | |
| CAGTGTTTAAGGTTCGATTACACAGGACAAGGAGGC | |
| AACGAAAACAATTTTCGCCGTACTTACGATTGCCAA | |
| CGAACGTGTCTGTATACAGGCTGATGCATCCGCTCT | |
| GGTGGTGGTGGCAATATCTCTAACACTGTTGAGGAG | |
| AGCCAAGGGCGAATTC | |
| Translation: | |
| MEGRRFAAVLILTICMLAPGTGTLLPRDRPSLCDLP | (SEQ ID NO:26) |
| ADSGSGTKAEKRIYYNSARKQCLRFDYTGQGGNENN | |
| FRRTYDCQRTCLYTG | |
| Toxin Sequence: | |
| Thr-Leu-Leu-Xaa3-Arg-Asp-Arg-Xaa3- | (SEQ ID NO:27) |
| Ser-Leu-Cys-Asp-Leu-Xaa3-Ala-Asp- | |
| Ser-Gly-Ser-Gly-Thr-Lys-Ala-Xaa1- | |
| Lys-Arg-Ile-Xaa5-Xaa5-Asn-Ser-Ala- | |
| Arg-Lys-Gln-Cys-Leu-Arg-Phe-Asp- | |
| Xaa5-Thr-Gly-Gln-Gly-Gly-Asn-Xaa1- | |
| Asn-Asn-Phe-Arg-Arg-Thr-Xaa5-Asp- | |
| Cys-Gln-Arg-Thr-Cys-Leu-Xaa5-Thr-# | |
| Name: C1722 | |
| Species: striatus | |
| Isolated: No | |
| Cloned: Yes | |
| DNA Sequence: | |
| GAATTCGCCCTTATGGAGGGACGTCGTTTTGCTGCT | (SEQ ID NO:28) |
| GTTCTGATCCTGACCCTCTGTATGCTTGCATCTGGG | |
| GCTGTCGCTGCGAGACCTAAGGATCGACCGAGTTAT | |
| TGCAATCTACCAGCGGACAGTGGGTCGGGCACAAAG | |
| CCTGAGCAGAGAATTTACTACAATAGCGCTAAAAAA | |
| CAGTGTGTAACGTTCACTTACAATGGAAAAGGAGGC | |
| AACGGCAACAATTTTAGCCGTACTAACGATTGCCGA | |
| CAAACGTGTCAGTATCCAGTTGGCTGAAATAACTGC | |
| GCTGACTGGTGTGGCGACATCCCTAACACTGTTGAG | |
| GAGAGCCAAGGGCGAATTC | |
| Translation: | |
| MEGRRFAAVLILTLCMLASRAVAARPDRPSYCNLPA | (SEQ ID NO:29) |
| DSGSGTKPEQRIYYNSAKKQCVTFTYNGKGGNGNNF | |
| SRTNDCRQTCQYPVG | |
| Toxin Sequence: | |
| Ala-Arg-Xaa3-Lys-Asp-Arg-Xaa3-Ser- | (SEQ ID NO:30) |
| Xaa5-Cys-Asn-Leu-Xaa3-Ala-Asp-Ser- | |
| Gly-Ser-Gly-Thr-Lys-Xaa3-Xaa1-Gln- | |
| Arg-Ile-Xaa5-Xaa5-Asn-Ser-Ala-Lys- | |
| Lys-Gln-Cys-Val-Thr-Phe-Thr-Xaa5- | |
| Asn-Gly-Lys-Gly-Gly-Asn-Gly-Asn-Asn- | |
| Phe-Ser-Arg-Thr-Asn-Asp-Cys-Arg-Gln- | |
| Thr-Cys-Gln-Xaa5-Xaa3-Val-# | |
| Name: C1723 | |
| Species: striatus | |
| Isolated: No | |
| Cloned: Yes | |
| DNA Sequence: | |
| GAATTCGCCCTTATGGAGGGACGTCGTTTTGCTGCT | (SEQ ID NO:31) |
| GTTCTGATCCTGACCCTCTGTATGCTTGCATCTCGG | |
| GCTGTCGCTGCGAGACCTAAGGATCGACCGAGTTAT | |
| TGCAATCTACCAGCGGACAGTGGGTCGGGCACAAAG | |
| CCTGAGCAGAGAATTTACTACAATAGCGCTAAAAAA | |
| CAGTGTGTAACGTTCACTTACAGTGGAAAAGGAGGC | |
| AACGGCAACAATTTTAGCCGTACTAACGATTGCCGA | |
| CAAACGTGTCAGTATCCAGCTGGCTGAAATAACTGC | |
| GCTGACTGTTGTGGCGACATCCCTAACACTGTTGAG | |
| GAGAGCCAAGGGCGAATTC | |
| Translation: | |
| MEGRRFAAVLILTLCMLASRAVAARPKDRPSYCNLP | (SEQ ID NO:32) |
| ADSGSGTKPEQRIYYNSAKKQCVTFTYSGKGGNGNN | |
| GSRTNDCRQTCQYPAG | |
| Toxin Sequence: | |
| Ala-Arg-Xaa3-Lys-Asp-Arg-Xaa3-Ser- | (SEQ ID NO:33) |
| Xaa5-Cys-Asn-Leu-Xaa3-Ala-Asp-Ser- | |
| Gly-Ser-Gly-Thr-Lys-Xaa3-Xaa1-Gln- | |
| Arg-Ile-Xaa5-Xaa5-Asn-Ser-Ala-Lys- | |
| Lys-Gln-Cys-Val-Thr-Phe-Thr-Xaa5- | |
| Ser-Gly-Lys-Gly-Gly-Asn-Gly-Asn-Asn- | |
| Phe-Ser-Arg-Thr-Asn-Asp-Cys-Arg-Gln- | |
| Thr-Cys-Gln-Xaa5-Xaa3-Ala-# | |
| Name: C1726 | |
| Species: striatus | |
| Isolated: No | |
| Cloned: Yes | |
| DNA Sequence: | |
| GAATTCGCCCTTATGGAGGGACGTCGTTTTGCTGCT | (SEQ ID NO:34) |
| GTTCTGATCCTGACCCTCTGTATGCTTGCATCTGGG | |
| GCTGTCGCTGCGAAACCTAAGAGTCGGCCGAGTTAT | |
| TGCAATCTACCAGCGGACAGTGGGTCGGGCACAAAG | |
| TCTGAGAAGAGAATTTACTACAATAGCGCTAGAAAA | |
| CAGTGTTTAGGGTTCACTTACCATGGAGAAGGAGGC | |
| AACGCAAACAACTTTCTCCATACTTTCGATTGCCAA | |
| CATACGTGTCTGTATACAGTCTGATGCATCCGCTCT | |
| GGCGGTGGTGGCAATATCTCTAACACTGTTGAGGAG | |
| AGCCAAGGGCGAATTC | |
| Translation: | |
| MEGRRFAAVLILTLCMLASGAVAAKPKSRPSYCNLP | (SEQ ID NO:35) |
| ADSGSGTKSEKRIYYNSARKQCLGFTYHGEGGNANN | |
| GLHTFDCQHTCLYTV | |
| Toxin Sequence: | |
| Ala-Lys-Xaa3-Lys-Ser-Arg-Xaa3-Ser- | (SEQ ID NO:36) |
| Xaa5-Cys-Asn-Leu-Xaa3-Ala-Asp-Ser- | |
| Gly-Ser-Gly-Thr-Lys-Ser-Xaa1-Lys- | |
| Arg-Ile-Xaa5-Xaa5-Asn-Ser-Ala-Arg- | |
| Lys-Gln-Cys-Leu-Gly-Phe-Thr-Xaa5- | |
| His-Gly-Xaa1-Gly-Gly-Asn-Ala-Asn- | |
| Asn-Phe-Leu-His-Thr-Phe-Asp-Cys-Gln- | |
| His-Thr-Cys-Leu-Xaa5-Thr-Val-\hat{ } | |
| Name: C1728 | |
| Species: consors | |
| Isolated: No | |
| Cloned: Yes | |
| DNA Sequence: | |
| GAATTCGCCCTTATGGAGGGACGTCGTTTTGCTGCT | (SEQ ID NO:37) |
| GTTCTGATCCTGACCCTCTGTATGCTTGCATCTGGG | |
| GCTGTCGCTGCGAAACCTAAGAGTCGGCCGAGTTAT | |
| TGCAATCTACCAGCGGACAGTGGGTCGGGCACAAAG | |
| TCTGAGAAGAGAATTTACTACAATAGCGCTAGAAAA | |
| CAGTGTTTAAGGTTCACTTACCATGGAGAAAGGGAA | |
| GGGCCAAACGNCAAAAACAATTTTCTCCATACTTTC | |
| GATTGCCAACATACGTGTCTGTATACAGTCTGATGC | |
| ATCCGCTCTGGCGGTGGTGGCAATATCTCTAACACT | |
| GTTGAGGAGAGCCAAGGGCGAATTC | |
| Translation: | |
| MEGRRFAAVLILTLCMLASGAVAAKPKSRPSYCNLP | (SEQ ID NO:38) |
| ADSGSGTKSEKRIYYNSARKQCLRFTYHGEREGPNX | |
| KNNFLHTFDCQHTCLYTV | |
| Toxin Sequence: | |
| Ala-Lys-Xaa3-Lys-Ser-Arg-Xaa3-Ser- | (SEQ ID NO:39) |
| Xaa5-Cys-Asn-Leu-Xaa3-Ala-Asp-Ser- | |
| Gly-Ser-Gly-Thr-Lys-Ser-Xaa1-Lys- | |
| Arg-Ile-Xaa5-Xaa5-Asn-Ser-Ala-Arg- | |
| Lys-Gln-Cys-Leu-Arg-Phe-Thr-Xaa5- | |
| His-Gly-Xaa1-Arg-Xaa1-Gly-Xaa3-Asn- | |
| Xaa6-Lys-Asn-Asn-Phe-Leu-His-Thr- | |
| Phe-Asp-Cys-Gln-His-Thr-Cys-Leu- | |
| Xaa5-Thr-Val-\hat{ } | |
| Name: C1733 | |
| Species: consors | |
| Isolated: No | |
| Cloned: Yes | |
| DNA Sequence: | |
| GAATTCGCCCTTATGGAGGGACGTCGTTTTGCTGCT | (SEQ ID NO:40) |
| GTTCTGATCCTGACCCTCTGTATGCTTGCATCTGGG | |
| GCTGTCGCTGCGAAACCTAAGAGTCGGCCGAGTTAT | |
| TGCAATCTACCAGCGGACAGTGGGTCGGGCACAAAG | |
| TCTGAGAAGAGAATTTACTACAATAGCGCTAGAAAA | |
| CAGTGTTTAAGGTTCACTTACCATGGAGAAGGAGGC | |
| AACGCAAACAATTTTCTCCATACTTTCGATTGCCAA | |
| CATACGTGTCTGTATACAGTCTGATGCATCCGCTCT | |
| GGCGGTGGTGGCAATATCTCTAACACTGTTGAGGAG | |
| AGCCAAGGGCGAATTC | |
| Translation: | |
| MEGRRFAAVLILTLCMLASGAVAAKPKSRPSYCNLP | (SEQ ID NO:41) |
| ADSGSGTKSEKRIYYNSARKQCLRFTYHGEGGNANN | |
| GLHTFDCQHTCLYTV | |
| Toxin Sequence: | |
| Ala-Lys-Xaa3-Lys-Ser-Arg-Xaa3-Ser- | (SEQ ID NO:42) |
| Xaa5-Cys-Asn-Leu-Xaa3-Ala-Asp-Ser- | |
| Gly-Ser-Gly-Thr-Lys-Ser-Xaa1-Lys- | |
| Arg-Ile-Xaa5-Xaa5-Asn-Ser-Ala-Arg- | |
| Lys-Gln-Cys-Leu-Arg-Phe-Thr-Xaa5- | |
| His-Gly-Xaa1-Gly-Gly-Asn-Ala-Asn- | |
| Asn-Phe-Leu-His-Thr-Phe-Asp-Cys-Gln- | |
| His-Thr-Cys-Leu-Xaa5-Thr-Val-\hat{ } | |
| Name: C1736 | |
| Species: consors | |
| Isolated: No | |
| Cloned: Yes | |
| DNA Sequence: | |
| GAATTCGCCCTTATGGAGGGACGTCGTTTTGCTGCT | (SEQ ID NO:43) |
| GTTCTGATCCTGACCCTCTGTATGCTTGCATCTGGG | |
| GCTGTCGCTGCGAGACCTAAGGATCGACCGAGTTAT | |
| TGCAATCTACCAGCGGACAGTGGGTCGGGCACAAAG | |
| CCTGAGCAGAGAATTTACTACAATAGCGCTCGAAAA | |
| CAGTGTTTAACGTTCACTTACAATGGAAAAGGAGGC | |
| AACGAAAACAATTTTATCCATACTTACGATTGCCGA | |
| CGAACGTGTCAGTATACAGTCTGATGCATCCGCTCT | |
| GGCGGTGGTGGCAATATCTCTAACACTGTTGAGGAG | |
| AGCCAAGGGCGAATTC | |
| Translation: | |
| MEGRRFAAVLILTLCMLASGAVAARPKDRPSYCNLP | (SEQ ID NO:44) |
| ADSGSGTKPEQRIYYNSARKQCLTFTYNGKGGNENN | |
| FIHTYDCRRTCQYTV | |
| Toxin Sequence: | |
| Ala-Arg-Xaa3-Lys-Asp-Arg-Xaa3-Ser- | (SEQ ID NO:45) |
| Xaa5-Cys-Asn-Leu-Xaa3-Ala-Asp-Ser- | |
| Gly-Ser-GlyThr-Lys-Xaa3-Xaa1-Gln- | |
| Arg-Ile-Xaa5-Xaa5-Asn-Ser-Ala-Arg- | |
| Lys-Gln-Cys-Leu-Thr-Phe-Thr-Xaa5- | |
| Asn-Gly-Lys-Gly-Gly-Asn-Xaa1-Asn- | |
| Asn-Phe-Ile-His-Thr-Xaa5-Asp-Cys- | |
| Arg-Arg-Thr-Cys-Gln-Xaa5-Thr-Val-\hat{ } | |
| Name: C1741 | |
| Species: catus | |
| Isolated: No | |
| Cloned: Yes | |
| DNA Sequence: | |
| GAATTCGCCCTTATGGAGGGACGTCGTTTTGCTGCT | (SEQ ID NO:46) |
| GTTCTGATCCTGACCACCTGTATGCTTGCACCTGGG | |
| ACTGGTACTTTGATACCTCGGGGTCGACCGAGTTAT | |
| TGCAATCTACGAAAGGACAGTGGGTCGTGCACAAGG | |
| TCTGAGCAGAGCACAGAGTCTGAGTACAGATATTAC | |
| TTCAGTACACATTATAATGAGTGTAGAATGTTCACT | |
| TACAAAGGATGTGGAGGCAACCGCAACAATTTTATC | |
| CATATGCACGATTGTCGACAAAAGTGTGTGTATACA | |
| GTTGGCTGAAATAATTGCGCTGACTGTTGTGGCGAC | |
| ATCCCTAACACTGTTGAGGAGATCCAAGGGCGAATT | |
| C | |
| Translation: | |
| MEGRRFAAVLILTTCMLAPGTGTLIPRGRPSYCNLR | (SEQ ID NO:47) |
| KDSGSCTRSEQSTESEYRYYFSTHYNECRMFTYKGC | |
| GGNRNNFIHMHDCRQKCVYTVG | |
| Toxin Sequence: | |
| Thr-Leu-Ile-Xaa3-Arg-Gly-Arg-Xaa3- | (SEQ ID NO:48) |
| Ser-Xaa5-Cys-Asn-Leu-Arg-Lys-Asp- | |
| Ser-Gly-Ser-Cys-Thr-Arg-Ser-Xaa1- | |
| Gln-Ser-Thr-Xaa1-Ser-Xaa1-Xaa5-Arg- | |
| Xaa5-Xaa5-Phe-Ser-Thr-His-Xaa5-Asn- | |
| Xaa1-Cys-Arg-Met-Phe-Thr-Xaa5-Lys- | |
| Gly-Cys-Gly-Gly-Asn-Arg-Asn-Asn-Phe- | |
| Ile-His-Met-His-Asp-Cys-Arg-Gln-Lys- | |
| Cys-Val-Xaa5-Thr-Val-# | |
| Name: C1742 | |
| Species: catus | |
| Isolated: No | |
| Cloned: Yes | |
| DNA Sequence: | |
| GAATTCGCCCTTATGGAGGGACGTCGTTTTGCTGCT | (SEQ ID NO:49) |
| GTTCTGATCCTGACCATCTGTATGCTTGCACCTGGG | |
| ACTGGTACTTTGATACCTCGGGATCGACCGAGTTAT | |
| TGCAATCTACCAGCGGACAGTGGGTCGGGCACAAAT | |
| CGTGAGCAGAGAATTTACTACAATAGCGCTAGAAAA | |
| CAGTGTTTAACGTTCCCTTACAAAGGAAAAGGAGGC | |
| AACGCAAACAATTTTAGCCGTACTAACGATTGCCGA | |
| CGAACGTGTCAGTATCCAGCAGCTGGCTGAAATAAT | |
| TGCGCTGACTGTTGTGGCGACATCCCTAACACTGTT | |
| GAGGAGAGCCAAGGGCGAATTC | |
| Translation: | |
| MEGRRFAAVLILTICMLAPGTGTLIPRDRPSYCNLP | (SEQ ID NO:50) |
| ADSGSGTNREQRIYYNSARKQCLTFPYKGKGGNANN | |
| FSRTNDCRRTCQYPAAG | |
| Toxin Sequence: | |
| Thr-Leu-Ile-Xaa3-Arg-Asp-Arg-Xaa3- | (SEQ ID NO:51) |
| Ser-Xaa5-Cys-Asn-Leu-Xaa3-Ala-Asp- | |
| Ser-Gly-Ser-Gly-Thr-Asn-Arg-Xaa1- | |
| Gln-Arg-Ile-Xaa5-Xaa5-Asn-Ser-Ala- | |
| Arg-Lys-Gln-Cys-Leu-Thr-Phe-Xaa3- | |
| Xaa5-Lys-Gly-Lys-Gly-Gly-Asn-Ala- | |
| Asn-Asn-Phe-Ser-Arg-Thr-Asn-Asp-Cys- | |
| Arg-Arg-Thr-Cys-Gln-Xaa5-Xaa3-Ala- | |
| Ala-# | |
| Name: C1754 | |
| Species: betulinus | |
| Isolated: No | |
| Cloned: Yes | |
| DNA Sequence: | |
| GAATTCGCCCTTATGGAGGGACGTCGTTTTGCTGCT | (SEQ ID NO:52) |
| GTTCTGATCCTGACCATCTGTCTGCTGGCACTTGGG | |
| GACGGCGCTTCCAGACGTCAGGGTCCGCCAACAAAT | |
| AAGTGTTCTCGTGATGTTGCCCTAAGTAAGTGTTAC | |
| CAACCCAGGGATCCGGGGCCGTGTAATGCTTTCTTT | |
| GAGAAGTATTACTTCAACAGGTATATCAACACTTGC | |
| GAAAAATTCACTTACGGAGGATGTCAAGGCAACGAC | |
| AACAGATTTAACACCGTGGAGGACTGCCTAGGATGT | |
| TGTCTACTGAGTGTATGCAGACAACCAGCGGAAACG | |
| GGACCGTGCAGAGGGTATTTCCCGAAATATTACTTC | |
| GATATGGATTCTTACGAGTGCAAACAGTTCATTTAC | |
| GGAGGATGCGGTGGCAACGACAACAATTTTGGCACT | |
| GTCGAAGAGTGTCAAGGCAGATGTGGGCCGAAATAA | |
| CTGCGCTGATTGTTGTGGCGACATCCCTAACACTGT | |
| TGAGGAGAGCCAAGGGCGAATTC | |
| Translation: | |
| MEGRRFAAVLILTICLLALGDGASRRQGPPTNKCSR | (SEQ ID NO:53) |
| DVALSKCYQPRDPGPCNAFFEKYYFNRYINTCEKFT | |
| YGGCQGNDNRFNTVEDCLGCCLLSVCRQPAETGPCR | |
| GYFPKYYFDMDSYECKQFIYGGCGGNDNNFGTVEEC | |
| QGRCGPK | |
| Toxin Sequence: | |
| Ala-Ser-Arg-Arg-Gln-Gly-Xaa3-Xaa3- | (SEQ ID NO:54) |
| Thr-Asn-Lys-Cys-Ser-Arg-Asp-Val-Ala- | |
| Leu-Ser-Lys-Cys-Xaa5-Gln-Xaa3-Arg- | |
| Asp-Xaa3-Gly-Xaa3-Cys-Asn-Ala-Phe- | |
| Phe-Xaa1-Lys-Xaa5-Xaa5-Phe-Asn-Arg- | |
| Xaa5-Ile-Asn-Thr-Cys-Xaa1-Lys-Phe- | |
| Thr-Xaa5-Gly-Gly-Cys-Gln-Gly-Asn- | |
| Asp-Asn-Arg-Phe-Asn-Thr-Val-Xaa1- | |
| Asp-Cys-Leu-Gly-Cys-Cys-Leu-Leu-Ser- | |
| Val-Cys-Arg-Gln-Xaa3-Ala-Xaa1-Thr- | |
| Gly-Xaa3-Cys-Arg-Gly-Xaa5-Phe-Xaa3- | |
| Lys-Xaa5-Xaa5-Phe-Asp-Met-Asp-Ser- | |
| Xaa5-Xaa1-Cys-Lys-Gln-Phe-Ile-Xaa5- | |
| Gly-Gly-Cys-Gly-Gly-Asn-Asp-Asn-Asn- | |
| Phe-Gly-Thr-Val-Xaa1-Xaa1-Cys-Gln- | |
| Gly-Arg-Cys-Gly-Xaa3-Lys-\hat{ } | |
| Name: C1756 | |
| Species: betulinus | |
| Isolated: No | |
| Cloned: Yes | |
| DNA Sequence: | |
| GAATTCGCCCTTATGGAGGGACGTCGTTTTGCTGCT | (SEQ ID NO:55) |
| GTTCTGATCCTGACCATCTGTCTGCTTGCTCTTGGG | |
| GCTGGCGCTTCCAGACGTCAGGGTCCGCCAACAAAC | |
| AAGTGTTCTCGTGATGTTGCCCTAAGTAAGTGTTAC | |
| CAACCCAGGGATCCGGGGCCGTGTTATGCTTTGTTG | |
| GAGAGGTATTACTTCAACAGGTATATCAACACTTGC | |
| GAAAAATTCACTTACGGAGGATGTCAAGGCAACGAC | |
| AACAGATTTAACACCATGGAGGATTGCCTAGGATGT | |
| TGTCAACTGAGTGTATGCAGACAACCAGCGGAACCG | |
| GGACTGTGCAGAGGGTATTTCCTGAGACATTACTTC | |
| GATATGGATTTTAACGAGTGCAAAGAGTTCATTTAC | |
| GGAGGATGCGGTGGCAACGACAACAATTTTCTCACT | |
| GACGAAGAGTGTCAAGGCAGATGTGGGCCGGAATAA | |
| CTGCGCTGATTGTTGTGGCGACATCCCTAACACTGT | |
| TGAGGAGAGCCAAGGGCGAATTC | |
| Translation: | |
| MEGRRFAAVLILTICLLALGAGASRRQGPPTNKCSR | (SEQ ID NO:56) |
| DVALSKCYQPRDPGPCYALLERYYFNRYTNTCEKFT | |
| YGGCQGNPNRFNTMEDCLGCCQLSVCRQPAEPGLCR | |
| GYFLRHYFDMDFNECKEGIYGGCGGNDNNFLTDEEC | |
| QGRCGPE | |
| Toxin Sequence: | |
| Ser-Arg-Arg-Gln-Gly-Xaa3-Xaa3-Thr- | (SEQ ID NO:57) |
| Asn-Lys-Cys-Ser-Arg-Asp-Val-Ala-Leu- | |
| Ser-Lys-Cys-Xaa5-Gln-Xaa3-Arg-Asp- | |
| Xaa3-Gly-Xaa3-Cys-Xaa5-Ala-Leu-Leu- | |
| Xaa1-Arg-Xaa5-Xaa5-Phe-Asn-Arg-Xaa5- | |
| Ile-Asn-Thr-Cys-Xaa1-Lys-Phe-Thr- | |
| Xaa5-Gly-Gly-Cys-Gln-Gly-Asn-Asp- | |
| Asn-Arg-Phe-Asn-Thr-Met-Xaa1-Asp- | |
| Cys-Leu-Gly-Cys-Cys-Gln-Leu-Ser-Val- | |
| Cys-Arg-Gln-Xaa3-Ala-Xaa1-Xaa3-Gly- | |
| Leu-Cys-Arg-Gly-Xaa5-Phe-Leu-Arg- | |
| His-Xaa5-Phe-Asp-Met-Asp-Phe-Asn- | |
| Xaa1-Cys-Lys-Xaa1-Phe-Ile-Xaa5-Gly- | |
| Gly-Cys-Gly-Gly-Asn-Asp-Asn-Asn-Phe- | |
| Leu-Thr-Asp-Xaa1-Xaa1-Cys-Gln-Gly- | |
| Arg-Cys-Gly-Xaa3-Xaa1-\hat{ } | |
| Name: C1757 | |
| Species: betulinus | |
| Isolated: No | |
| Cloned: Yes | |
| DNA Sequence: | |
| GAATTCGCCCTTATGGAGGGACGTCGTTTTGCTGCT | (SEQ ID NO:58) |
| GCTCTGGTCCTGACCATTTGTCTGCTTGCACTTGGG | |
| GCTGACGCTTCCAGACGTCAGGATCTGTGCAGTCTA | |
| CCAGCGGAAAAGGGACCGTGCTTAGCATATATGCCG | |
| AGATTTTACTTCGATTGGGATTCTAAGTTATGCAAA | |
| GTGTTCATTTACGGAGGATGTCAGGGCAACGACAAC | |
| AATTTTCACAGTTCCACGGACTGTTACGACACATGT | |
| TGGTTTGAATAACTGCGCTGATTGTTGTGGCGACAT | |
| CCCTAACACTGTTGAGGAGAGCCAAGGGCGAATTC | |
| Translation: | |
| MEGRRFAAALVLTICLLALGADASRRQDLCSLPAEK | (SEQ ID NO:59) |
| GCLAYMPRFYFDWDSKLCKVFIYGGCQGNDNNFHSS | |
| TDCYDTCWFE | |
| Toxin Sequence: | |
| Ser-Arg-Arg-Gln-Asp-Leu-Cys-Ser-Leu- | (SEQ ID NO:60) |
| Xaa3-Ala-Xaa1-Lys-Gly-Xaa3-Cys-Leu- | |
| Ala-Xaa5-Met-Xaa3-Arg-Phe-Xaa5-Phe- | |
| Asp-Xaa4-Asp-Ser-Lys-Leu-Cys-Lys- | |
| Val-Phe-Ile-Xaa5-Gly-Gly-Cys-Gln- | |
| Gly-Asn-Asp-Asn-Asn-Phe-His-Ser-Ser- | |
| Thr-Asp-Cys-Xaa5-Asp-Thr-Cys-Xaa4- | |
| Phe-Xaa1-\hat{ } | |
| Name: C1758 | |
| Species: betulinus | |
| Isolated: No | |
| Cloned: Yes | |
| DNA Sequence: | |
| GAATTCGCCCTTATGGAGGGACGTCGTTTTGCTGCT | (SEQ ID NO:61) |
| GTTCTGATCCTGACCATCTGTCTGCTTGCACTTGGG | |
| GCTGGCGCTTCCAGACGTCAGGATCTGTGCAGTCTA | |
| CCAGCGGAAAAGGGACCGTGCTTAGCATATATGCCG | |
| AGATTTTACTTCGATTGGGATTCTAAGTTATGCAAA | |
| GTGTTCATTTACGGAGGATGTCAGGGCAACGACAAC | |
| AATTTTCACAGTTCCTCGGACTGTTACGACACATGT | |
| TGGTTTGAATAACTGCGCTGATTGTTGTGGCGACAC | |
| CCCTAACACTGTTGAGGAGAGCCAAGGGCGAATTC | |
| Translation: | |
| MEGRRFAAVLILTICLLALGAGASRRQDLCSLPAEK | (SEQ ID NO:62) |
| GPCLAYMPRFYFDWDSKLCKVFIYGGCQGNDNNFHS | |
| SSDCYDTCWFE | |
| Toxin Sequence: | |
| Ser-Arg-Arg-Gln-Asp-Leu-Cys-Ser-Leu- | (SEQ ID NO:63) |
| Xaa3-Ala-Xaa1-Lys-Gly-Xaa3-Cys-Leu- | |
| Ala-Xaa5-Met-Xaa3-Arg-Phe-Xaa5-Phe- | |
| Asp-Xaa4-Asp-Ser-Lys-Leu-Cys-Lys- | |
| Val-Phe-Ile-Xaa5-Gly-Gly-Cys-Gln- | |
| Gly-Asn-Asp-Asn-Asn-Phe-His-Ser-Ser- | |
| Ser-Asp-Cys-Xaa5-Asp-Thr-Cys-Xaa4- | |
| Phe-Xaa1-\hat{ } | |
| Name: C1760 | |
| Species: betulinus | |
| Isolated: No | |
| Cloned: Yes | |
| DNA Sequence: | |
| GAATTCGCCCTTATGGAGGGACGTCGTTTTGCTGCT | (SEQ ID NO:64) |
| GTTCTGATCCTGACCATCTGTCTGCTGGCACTTGGG | |
| GCTGGCGCTTCCAAACGTCAGGGTCCGCCAACAAAC | |
| AAGTGTTCTCGTGATGTCGCCCTAAGTAAGTGTTAC | |
| CAACCCAGGGATCCGGGGCCGTGTTATGCTTCGTTT | |
| CGCATGTATTACTTCAACAGGTATATCAACACTTGC | |
| GAAAAATTCACTTACGGAGGATGTCAAGGCAACGAC | |
| AACAGATTTAACACCGTGGAGGACTGCCTAGGATGT | |
| TGTCAACTGAGTGTATGCAGACAACCAGCGGAACCG | |
| GGACTGTGCAAAGGGTATTTCCCGAGACATTACTTC | |
| GATATGGATTCTTACGAGTGCAAAACGTTCATTTAC | |
| GGAGGATGCGGTGGCAACGACAACAATTTTCTCACT | |
| GACGGAGAGTGTCAAGGCAGATGTGGGCCGGAATAA | |
| CTGCGCTGATTGTTGTGGCGACATCCCTAACACTGT | |
| TGAGGAGAGCCAAGGGCGAATTC | |
| Translation: | |
| MEGRRFAAVLILTICLLALGAGASKRQGPPTNKCSR | (SEQ ID NO:65) |
| DVALSKCYQPRDPGPCYASFRMYYFNRYINTCEKFT | |
| YGGCQGNDNRFNTVEDCLGCCQLSVCRQPAEPGLCK | |
| GYFPRHYFDMDSYECKTFIYGGCGGNDNNFLTDGEC | |
| QGRCGPE | |
| Toxin Sequence: | |
| Ser-Lys-Arg-Gln-Gly-Xaa3-Xaa3-Thr- | (SEQ ID NO:66) |
| Asn-Lys-Cys-Ser-Arg-Asp-Val-AIa-Leu- | |
| Ser-Lys-Cys-Xaa5-Gln-Xaa3 -Arg-Asp- | |
| Xaa3-Gly-Xaa3-Cys-Xaa5-Ala-Ser-Phe- | |
| Arg-Met-Xaa5-Xaa5-Phe-Asn-Arg-Xaa5- | |
| Ile-Asn-Thr-Cys-Xaa1-Lys-Phe-Thr- | |
| Xaa5-Gly-Gly-Cys-Gln-Gly-Asn-Asp- | |
| Asn-Arg-Phe-Asn-Thr-Val-Xaa1-Asp- | |
| Cys-Leu-Gly-Cys-Cys-Gln-Leu-Ser-Val- | |
| Cys-Arg-Gln-Xaa3-Ala-Xaa1-Xaa3-Gly- | |
| Leu-Cys-Lys-Gly-Xaa5-Phe-Xaa3-Arg- | |
| His-Xaa5-Phe-Asp-Met-Asp-Ser-Xaa5- | |
| Xaa1-Cys-Lys-Thr-Phe-Ile-Xaa5-Gly- | |
| Gly-Cys-Gly-Gly-Asn-Asp-Asn-Asn-Phe- | |
| Leu-Thr-Asp-Gly-Xaa1-Cys-Gln-Gly- | |
| Arg-Cys-Gly-Xaa3-Xaa1-\hat{ } | |
| Name: C1768 | |
| Species: biliosus | |
| Isolated: No | |
| Cloned: Yes | |
| DNA Sequence: | |
| GAATTCGCCCTTATGGAGGGACGTCGTTTTGCTGCT | (SEQ ID NO:67) |
| GTTATGATCCTCACCATCTGCGTGCTTGCACTTAGG | |
| GCTGGCGCTTTCAGACTTCGTGGTTCGCGAGCTGAA | |
| CAGTGTTCTCGTTCTGAAGCCTTAGCAAAGTGTTAC | |
| CTTCCCAAGGATCCGGGGCCGTGTAAGGCATGGAAG | |
| TCCAGGTATTACTTCAACAGATACCTCAACACGTGC | |
| CAAGAGTTCAGTTACGGAGGATGTCGTGGCAACGCC | |
| AACAGATTTGACACCATGGAGGACTGCCAAGAATGT | |
| TGTCTACTGAGTGTATGCAGACAACCAGCGGAACCG | |
| GGGCTGTGCAAAGCGTATATGGAGAGATATTACTTT | |
| GATATGGATTCTTACGACTGTAAGCCGTTCATTTAC | |
| GGAGGATGTAACGGCAACGACAACAATTTTCTCACT | |
| TACATTGAGTGTTACGGCAGATGTGGGCCGGAATAA | |
| CTGCGCTGGTTGTTGTGCCGACACCCCTAACACTGT | |
| TGAGGAGAGCCAAGGGCGAATTC | |
| Translation: | |
| MEGRRFAAVMILTICVLALRAGAFRLRGSRAEQCSR | (SEQ ID NO:68) |
| SEALAKCYLPKDPGPCKAWKSRYYFNRYLNTCQEFS | |
| YGGCRGNANRFDTMEDCQECCLLSVCRQPAEPGLCK | |
| AYMERYYFDMDSYDCKPFIYGGCNGNIDNNFLTYIE | |
| CYGRCGPE | |
| Toxin Sequence: | |
| Phe-Arg-Leu-Arg-Gly-Ser-Arg-Ala- | (SEQ ID NO:69) |
| Xaa1-Gln-Cys-Ser-Arg-Ser-Xaa1-Ala- | |
| Leu-Ala-Lys-Cys-Xaa5-Leu-Xaa3-Lys- | |
| Asp-Xaa3-Gly-Xaa3-Cys-Lys-Ala-Xaa4- | |
| Lys-Ser-Arg-Xaa5-Xaa5-Phe-Asn-Arg- | |
| Xaa5-Leu-Asn-Thr-Cys-Gln-Xaa1-Phe- | |
| Ser-Xaa5-Gly-Gly-Cys-Arg-Gly-Asn- | |
| Ala-Asn-Arg-Phe-Asp-Thr-Met-Xaa1- | |
| Asp-Cys-Gln-Xaa1-Cys-Cys-Leu-Leu- | |
| Ser-Val-Cys-Arg-Gln-Xaa3-Ala-Xaa1- | |
| Xaa3-Gly-Leu-Cys-Lys-Ala-Xaa5-Met- | |
| Xaa1-Arg-Xaa5-Xaa5-Phe-Asp-Met-Asp- | |
| Ser-Xaa5-Asp-Cys-Lys-Xaa3-Phe-Ile- | |
| Xaa5-Gly-Gly-Cys-Asn-Gly-Asn-Asp- | |
| Asn-Asn-Phe-Leu-Thr-Xaa5-Ile-Xaa1- | |
| Cys-Xaa5-Gly-Arg-Cys-Gly-Xaa3-Xaa1-\hat{ } | |
| Name: C1769 | |
| Species: biliosus | |
| Isolated: No | |
| Cloned: Yes | |
| DNA Sequence: | |
| GAATTCGCCCTTATGGAGGGACGTCGTTTTGCTGCT | (SEQ ID NO:70) |
| GTACTGATCCTCACCATCTGCGTGCTTGCACTTAGG | |
| GCTGGCGCTTTCAGACTTCGTGGTTCGCGAGCTGAA | |
| GAGTGTTCTCGTTCTGAAGCCTTAGCAAAGTGTTAC | |
| CTTCCCAAGGATCCGGGGCCGTGTAAGGCATGGAAG | |
| TCCAGGTATTACTTCAACAGATACCTCAACACGTGC | |
| CAAGAGTTCAGTTACGGAGGATGTCGTGGCAACGCC | |
| AACAGATTTGACACCATGGAGGACTGCCAAGAATGT | |
| TGTCTACTGAGTGTATGCAGACAACCAGCGGAACCG | |
| GGGCTGTGCAAAGCGTATATGGAGAGATATTACTTT | |
| GATATGGATTCTTACGACTGTAAGCCGTTCATTTAC | |
| GGAGGATGTAACGGCAACGACAACAATTTTCTCACT | |
| TACATTGAGTGTTACGGCAGATGTGGGCCGGAATAA | |
| CTGCGCTGGTTGTTGTGCCGACACCCCTAACACTGT | |
| TGAGGAGAGCCAAGGGCGAATTC | |
| Translation: | |
| MEGRRFAAVLILTICVLALRAGAFRLRGSRAEECSR | (SEQ ID NO:71) |
| SEALAKCYLPKDPGPCKAWKSRYYFNRYLNTCQEFS | |
| YGGCRGNANRFDTMEDCQECCLLSVCRQPAEPGLCK | |
| AYMERYYFDMDSYDCKPFIYGGCNGNDNNFLTYIED | |
| CYGRCGPE | |
| Toxin Sequence: | |
| Phe-Arg-Leu-Arg-Gly-Ser-Arg-Ala- | (SEQ ID NO:72) |
| Xaa1-Xaa1-Cys-Ser-Arg-Ser-Xaa1-Ala- | |
| Leu-Ala-Lys-Cys-Xaa5-Leu-Xaa3-Lys- | |
| Asp-Xaa3-Gly-Xaa3-Cys-Lys-Ala-Xaa4- | |
| Lys-Ser-Arg-Xaa5-Xaa5-Phe-Asn-Arg- | |
| Xaa5-Leu-Asn-Thr-Cys-Gln-Xaa1-Phe- | |
| Ser-Xaa5-Gly-Gly-Cys-Arg-Gly-Asn- | |
| Ala-Asn-Arg-Phe-Asp-Thr-Met-Xaa1- | |
| Asp-Cys-Gln-Xaa1-Cys-Cys-Leu-Leu- | |
| Ser-Val-Cys-Arg-Gln-Xaa3-Ala-Xaa1- | |
| Xaa3-Gly-Leu-Cys-Lys-Ala-Xaa5-Met- | |
| Xaa1-Arg-Xaa5-Xaa5-Phe-Asp-Met-Asp- | |
| Ser-Xaa5-Asp-Cys-Lys-Xaa3-Phe-Ile- | |
| Xaa5-Gly-Gly-Cys-Asn-Gly-Asn-Asp- | |
| Asn-Asn-Phe-Leu-Thr-Xaa5-Ile-Xaa1- | |
| Cys-Xaa5-Gly-Arg-Cys-Gly-Xaa3-Xaa1-\hat{ } | |
| Name: C1790 | |
| Species: wittigi | |
| Isolated: No | |
| Cloned: Yes | |
| DNA Sequence: | |
| GAATTCGCCCTTATGGAGGGACGTCGTTTTGCTGCT | (SEQ ID NO:73) |
| GTTCTGATCCTGACCATCTGTATGCTTGCACTTGGG | |
| GCTGGTGCTTCGAGACTTCGGGATCAACTGAATAGA | |
| TGCAAACAACCAAAGGTAACGGGGCCGTGCAAAGGG | |
| GCGTTTCGGAGGTATTACTTTGATACAGATGTTCGC | |
| AACTGTAAACAGTTCACTTACGGAGGATGTAGAGGC | |
| AACGACAACAATTTTCTCACTTATGATGACTGTAAC | |
| GCCACATGTGGTCCGAAATAATTGCGCTGATTGTTG | |
| TGGCGACATCCCTAGCACTGCTGAGGAGAGCCACGC | |
| TGTGTGCTGATTGTTGTGGCGACATCCCTAACACTG | |
| TTGAGGAGAGCCAAGGGCGAATTC | |
| Translation: | |
| MEGRRFAAVLILTICMLALGAGASRLRDQLNRCKQP | (SEQ ID NO:74) |
| KVTGPCKGAFRRYYFDTDVRNCKQFTYGGCRGNDNN | |
| FLTYDDCNATCGPK | |
| Toxin Sequence: | |
| Ser-Arg-Leu-Arg-Asp-Gln-Leu-Asn-Arg- | (SEQ ID NO:75) |
| Cys-Lys-Gln-Xaa3-Lys-Val-Thr-Gly- | |
| Xaa3-Cys-Lys-Gly-Ala-Phe-Arg-Arg- | |
| Xaa5-Xaa5-Phe-Asp-Thr-Asp-Val-Arg- | |
| Asn-Cys-Lys-Gln-Phe-Thr-Xaa5-Gly- | |
| Gly-Cys-Arg-Gly-Asn-Asp-Asn-Asn-Phe- | |
| Leu-Thr-Xaa5-Asp-Asp-Cys-Asn-Ala- | |
| Thr-Cys-GLy-Xaa3-Lys-\hat{ } | |
| Name: D1008 | |
| Species: distans | |
| Isolated: No | |
| Cloned: Yes | |
| DNA Sequence: | |
| GAATTCGGCCATTACGGCCGGGATCTACACTCCGCC | (SEQ ID NO:76) |
| TGCTGACACGTCATCTACTCTCTGTCTCCCTGACTG | |
| CTGCCTTCAGTCGACCCTGCCGTCATCTCAGCGCAG | |
| ACTTGGTAAGAAGTGAAAACCTTTATCATGGAGAAA | |
| CTGATAACCCTGCTTCTTGTTGCTACTGTCCTATTG | |
| TTGACCCTGGTTCAAGCAATAGCAGAAAACACCTGC | |
| GAACAGCCACCGGAGCCAGGTAGGTGTGAGGGATAC | |
| TATCGTAGGTTTTTCTACAACTGGAGGTCGGGCCAG | |
| TGCGAGTCATTTGTCTATGGAGGTTGTGGTGGCAAC | |
| GACAACAATTTCCGGACCAACGCTGAATGTGAGGCT | |
| GCGTGCTCCAGTACCAACACTTATGCTATATCAGAA | |
| AACACCTGCGAACAGCCAGCGGAGCAAGGTAGGTGT | |
| ACGGAGTACGTTGTCAGGTTTTTCTACAACTGGAAG | |
| TCGGACAAGTGCGAGCCATTTGTCTATGGAGGTTGT | |
| GGTGGCAACAGCAACAGTTTCCTGACCATCACTGAA | |
| TGTGAGACTGCATGCTCCAGTGGAAACACTTATGAA | |
| GACGTATGCCAACTGCCAGCTGATCGAGGGGTATGT | |
| TCGGCGCACTTGCACATGTTTTACTATAACTCCTCA | |
| TCAGGACAGTGCCATCAGTTTATCTACAGAGGTTGT | |
| GGNGGTAACGGCAACAAATTTTCGACCATGAGGGAA | |
| TGTCAAGCTAAGTGCTCCAGAGGCGTAAAGTACTCA | |
| AAGGTCTTGCCAACAAAACAATGGAAATTTTTTAGA | |
| CGAAACGAGCCCCGATATTTGTTGGCATGAGTAAAA | |
| GAGGAAATTGANCAGCNCGGAGAGGTNCCNNACGGA | |
| GTNCCNGGGGTGCTTGGGGATTCAGGACNCCCTGCT | |
| GCTGCTGTTGCATGCGGCTGGAATCCTTGGCGTTNT | |
| TTGACAGTTTNCCCCAGANGAANCCTTTGCTTGACA | |
| TCNCGTTTTTTCCCTTTTTNTCTTTTTGTAGATCGT | |
| TTTTAACCTGTTGGNGTTTTCANTTAGAACAGCGNG | |
| TTTTCAGATGTNCAGTATCCANGTNTATGNGATNTC | |
| TTTGTCTGCCNGTCTGTCTGTCTGTCTGTCTGTNTN | |
| TCAATNTGAGATTTTAGTCAGAACAATATNCNTTTA | |
| AACATAAAGTTTTCAANCCAANGTATTTNTAGTTTT | |
| TCCCTTTTNTTTTACCNCTNTTATTTTTCCCCNTGA | |
| TTTNCCNTTAAACNTTGTTTTTCTAAAAAAAAAAAA | |
| AAAAAAAAAAAAAAAAAACATGTCGGCCGCCTCGGC | |
| CCTCGAGAAGCTTTCTAGA | |
| Translation: | |
| MEKLITLLLVATVLLLTLVQAIAENTCEQPPEPGRC | (SEQ ID NO:77) |
| EGYYRRFFYNWRSGQCESFVYGGCGGNDNNFRTNAE | |
| CEAACSSTNTYAISENTCEQPAEQGRCTEYVVRFFY | |
| NWKSDKCEPFVYGGCGGNSNSFLTITECETACSSGN | |
| TYEDVCQLPADRGVCSAHLHMFYYNSSSGQCHQFIY | |
| RGCGGNGNKFSTMRECQAKCRGVKYSKVLPTKQWKF | |
| FRRNEPRYLLA | |
| Toxin Sequence: | |
| Xaa1-Asn-Thr-Cys-Xaa1-Gln-Xaa3-Xaa3- | (SEQ ID NO:78) |
| Xaa1-Xaa3-Gly-Arg-Cys-Xaa1-Gly-Xaa5- | |
| Xaa5-Arg-Arg-Phe-Phe-Xaa5-Asn-Xaa4- | |
| Arg-Ser-Gly-Gln-Cys-Xaa1-Ser-Phe- | |
| Val-Xaa5-Gly-Gly-Cys-Gly-Gly-Asn- | |
| Asp-Asn-Asn-Phe-Arg-Thr-Asn-Ala- | |
| Xaa1-Cys-Xaa1-Ala-Ala-Cys-Ser-Ser- | |
| Thr-Asn-Thr-Xaa5-Ala-Ile-Ser-Xaa1- | |
| Asn-Thr-Cys-Xaa1-Gln-Xaa3-Ala-Xaa1- | |
| Gln-Gly-Arg-Cys-Thr-Xaa1-Xaa5-Val- | |
| Val-Arg-Phe-Phe-Xaa5-Asn-Xaa4-Lys- | |
| Ser-Asp-Lys-Cys-Xaa1-Xaa3-Phe-Val- | |
| Xaa5-Gly-Gly-Cys-Gly-Gly-Asn-Ser- | |
| Asn-Ser-Phe-Leu-Thr-Ile-Thr-Xaa1- | |
| Cys-Xaa1-Thr-Ala-Cys-Ser-Ser-Gly- | |
| Asn-Thr-Xaa5-Xaa1-Asp-Val-Cys-Gln- | |
| Leu-Xaa3-Ala-Asp-Arg-Gly-Val-Cys- | |
| Ser-Ala-His-Leu-His-Met-Phe-Xaa5- | |
| Xaa5-Asn-Ser-Ser-Ser-Gly-Gln-Cys- | |
| His-Gln-Phe-Ile-Xaa5-Arg-Gly-Cys- | |
| Gly-Gly-Asn-Gly-Asn-Lys-Phe-Ser- | |
| Thr-Met-Arg-Xaa1-Cys-Gin-Ala-Lys- | |
| Cys-Ser-Arg-Gly-Val-Lys-Xaa5-Ser- | |
| Lys-Val-Leu-Xaa3-Thr-Lys-Gln-Xaa4- | |
| Lys-Phe-Phe-Arg-Arg-Asn-Xaa1-Xaa3- | |
| Arg-Xaa5-Leu-Leu-Ala-\hat{ } | |
| Name: D1085 | |
| Species: distans | |
| Isolated: No | |
| Cloned: Yes | |
| DNA Sequence: | |
| GAATTCGGCCATTACGGCCGGGGATTCAAGCGACGA | (SEQ ID NO:79) |
| GGTAGGTGAAGTGAAGGCGAGACGTAGGTGCATTTC | |
| TCTACTGAGTGCTGCTGTGTTCTGCCTTGACCTGCC | |
| TCAGCCCGGTCAGAAATACAGTGGGCTACAACACGG | |
| AGGTTCTGGCGATGTCAAAACTGGGAGTCGTGCTGA | |
| TCACCTTTCTGCTTCTGTTTCCCCTGGGAACTGTTC | |
| AGCTGGGTGGAGATGACCGCTGCACACTGCAGTTAT | |
| CCCACGGCACCTGTCCAGGCTATTTCCCCCGCTGGT | |
| TCTATGACTCGGCCAGTGGCCAGTGCCAGAGCTTCA | |
| TATACAGCGGATGTAAAGGCAACGCCAACAACTTTC | |
| TGTCTGAGGAGGAATGCCGCCAGGCTTGCGTCAGCC | |
| GTTGACGTCAATTATACCACGTTCCTTCCTTCCGTC | |
| GTGATGGAGATTTCAATCTTGTCATCGGTAGAAGCC | |
| AGTCTCGTCGTTCTATGTGTATGACGTCACCGTTTT | |
| ATGGTGTGACGTCACCGTTTTATGGATGTGGCGTGG | |
| CCTGAAACTGAAAAGGTATAAAAACATCAGAAATCT | |
| ACT | |
| Translation: | |
| MSKLGVVLITFLLLFPLGTVQLGGDDRCTLQLSHGT | (SEQ ID NO:80) |
| CPGYFPRWFYDSASGQCQSFIYSGCKGNANNFLSEE | |
| ECRQACVSR | |
| Toxin Sequence: | |
| Gly-Gly-Asp-Asp-Arg-Cys-Thr-Leu-Gln- | (SEQ ID NO:81) |
| Leu-Ser-His-Gly-Thr-Cys-Xaa3-Gly- | |
| Xaa5-Phe-Xaa3-Arg-Xaa4-Phe-Xaa5-Asp- | |
| Ser-Ala-Ser-Gly-Gln-Cys-Gln-Ser-Phe- | |
| Ile-Xaa5-Ser-Gly-Cys-Lys-Gly-Asn- | |
| Ala-Asn-Asn-Phe-Leu-Ser-Xaa1-Xaa1- | |
| Xaa1-\hat{ } | |
| Name: Conkunitzin S1 | |
| Species: striatus | |
| Isolated: No | |
| Cloned: Yes | |
| DNA Sequence: | |
| GAATTCGCCCTTATGGAGGGACGTCGTTTTGCTGCT | (SEQ ID NO:82) |
| GTTCTGATCCTGACCATCTGTATGCTTGCACCTGGG | |
| ACTGGTACTTTGCTACCTCGGGATCGACCGAGTCTA | |
| TGCGATCTACCAGCGGACAGTGGGTCGGGCACAAAG | |
| GCTGAGAAGAGAATTTACTACAATAGCGCTAGAAAA | |
| CAGTGTTTAAGGTTCGATTACACAGGACAAGGAGGC | |
| AACGAAAACAATTTTCGCCGTACTTACGATTGCCAA | |
| CGAACGTGTCTGTATACAGGCTGATGCATCCGCTCT | |
| GGTGGTGGTGGCAATATCTCTAACACTGTTGAGGAG | |
| AGCCAAGGGCGAATTC | |
| Translation: | |
| MEGRRFAAVLILTICMLAPGTGTLLPRDRPSLCDLP | (SEQ ID NO:83) |
| ADSGSGTKAEKRIYYNSARKQCLRFDYTGQGGNENN | |
| FRRTYDCQRTCLYTG | |
| Toxin Sequence: | |
| Thr-Leu-Leu-Xaa3-Arg-Asp-Arg-Xaa3- | (SEQ ID NO:84) |
| Ser-Leu-Cys-Asp-Leu-Xaa3-Ala-Asp- | |
| Ser-Gly-Ser-Gly-Thr-Lys-Ala-Xaa1- | |
| Lys-Arg-Ile-Xaa5-Xaa5-Asn-Ser-Ala- | |
| Arg-Lys-Gln-Cys-Leu-Arg-Phe-Asp- | |
| Xaa5-Thr-Gly-Gln-Gly-Gly-Asn-Xaa1- | |
| Asn-Asn-Phe-Arg-Arg-Thr-Xaa5-Asp- | |
| Cys-Gln-Arg-Thr-Cys-Leu-Xaa5-Thr-# | |
| Name: ShKm1 | |
| Species: magus | |
| Isolated: No | |
| Cloned: Yes | |
| DNA Sequence: | |
| GAATTCGCCCTTGGCGATGAATGAACACTGCGTTTG | (SEQ ID NO:85) |
| CTGGCTTTGACGAAAACCAGTCCACGTCAGCAGAGG | |
| GAGAGAGAGAGAGAGACCTTAGGAGCTGTGTATTCT | |
| CCAGTCATCTGACGGCCCCATCCACGTCAGTGCTGT | |
| TTCTTCTATCAGCCAGTCCATATCAACGCTCTACCG | |
| TCTGTCAGCCAGTCCACATCAGCGCTCTACCATCTG | |
| TCAGCCTGTTTACGTCAGCACTGTCATCTGACTGTC | |
| CAGGTTAAAAAATGGAGGGACGTCGTTTTGCTGCTG | |
| TTCTGATCCTGACCATCTGTATGCTTGCACCTGGGA | |
| CTGGTCGACCGAGTGTATGCAATAAACCAGCGGACA | |
| AGGGGCCGTGCACAGGGTCTGAGAAGAGATTCTACT | |
| TCAGTACCTATCATAATGAGTGTAGAACGTTCAAAT | |
| ACGGAGGATGTGAAGGCAACGGCAACAATTTTATCC | |
| ATGTGTACAATTGTCGAAGAACGTGTGTGTATCCAC | |
| CTGGCTGAAATAACTGCGCTGACTGTTGTGGCGACA | |
| TCCCTAACACTGTTGAGGAGAGCCAAGCTGTGGGCT | |
| TCTACAGATCCTTTCTACAGCGTCTAGTCTGTGTTC | |
| TGTATCTCTGTGGATCCTTTTAATGGTATCTGTTTT | |
| GGGTATCTCTATAGATACTTTACAGAGGGTCTGTTT | |
| TGTGTTTCTGTACAGATCCTTTTCAAAGTGTCTGTT | |
| TTGTGTATCTCTACAGGTCCTTTTCAAAGTGTCTGT | |
| TTTGTGTATCTCTACAGATCCTTTTCGTAGTGTCTG | |
| TTTTGTGTATCGCTACAGATCCTTTTCAAAGTGTTT | |
| TGTGTATCTCTACAGATCCTTTTCAAATTGTTTTGT | |
| GTATCTCTACAGATCCTTTTCAAAGTGTCTGTTTGT | |
| GTACCTCTACAGATACTTCCCATAGAGTCTGTTATA | |
| GAGTCTATAAATCCTTTCTATAGCATCTCTTTTTAT | |
| GCGCCTGTACATATCCGTTTCACTGCATCTATTTTG | |
| TGCATCTCTATAGATCCTTTCCATAGTGTCTGTACT | |
| CTGTATTCCAAAAGATTCTTTCCGTGGTGTCTTTCT | |
| TGTGCACCACTACGCATCCTTTCCATAGTATCTGTC | |
| TTGATTATCTCCCCACACTCTTTACTCGGTGCTTTC | |
| TCCTTATTTCACCCCCTTCTCATTCTGTGTTGATTC | |
| TCCAATCCTATCTTTCTCCTGCTTCCTTTTTTCCCT | |
| CCACTTGTAGTACTTGTTCGTCTTCTGAGCTCTTTT | |
| TTATTTTTTACTTCTCCTGTTCTTAAAAAAGGGCAT | |
| TGAATAAAATTATGTCAGATGAATCAATGTAAATGT | |
| TATCTACAGACAAGAGAGAACCTTTTTTTTTTGGTA | |
| CGTCTTCCAAACGACACACATATGAATCTGTGGAAA | |
| AAGTTATTTACAGATAAGAAGCGAGTTTCTTTTTTT | |
| AAACATATACTCCAAAATATGTACACGATCGCCATG | |
| TGCCCTACCAGTCTGACTGTCATCTTTTGATTTTTG | |
| ATATTAGGATGCGTTCTTTTTTTTTTTTTTTAACTA | |
| AAACTATTCTAGCCAAACATCTTCAACACTATCTAA | |
| ATGTCATAAAGAATGCATGAATCGTTTAACACTATT | |
| TAGAGAATAAGATTACTTTAGGTATCAGGCTTTATA | |
| TGTATGATAGTTTTGTTATTCTATCCATTGGCATCG | |
| TGCCTGCTGAACCAGATATACACATCACTTTTTTTA | |
| TTAGAAAGTAGTCGTCAGAAATTTGGAAAAAAAAGT | |
| CAAACGGCTATTTTGTATGTTTTCCAGAATTTATGG | |
| TTGAGACGCATTTACATTGTTCTGTTTGCATAATGT | |
| TTGCTTGGCTTCGCCAGTGTGTATTTGTAATAGCCT | |
| GGTGCGGCTTGAAAACCCCAAGGCTGAAGTGTCGCC | |
| AAATCTCGTGGAGCAAAATCAGGAAGGAGGAGGACA | |
| GGGCGACCTTAAAACTCCATCTACTCAGCACTGTTT | |
| GGATTGCCTCATCATTTCCTTTCAATACTTTTCGTT | |
| TTAAACGTTGGTGTCTATTGGATTTTTCTTCATTAT | |
| TCACCACTTAATCTACGGTGGAGGATCTTTGTTCCC | |
| GTCTTTACCTCTTTGTCTGCTTTGTACGTGCTTCTG | |
| TTCTTCTTTGAATCGAACAGTAAATGTCTGCTTTGT | |
| CCATTTAGGGGAGTGGTGTCCCCTTGTTTTGAAATC | |
| TTGTGAACTGGTTTCTGCCAATTAAACCTATTTTCT | |
| CTGTAGAAGCGAAAAAAAAAAAAAAAAAAAGTACTC | |
| TGCGTTGTTACTCGGCTTAAGGGCGAATTC | |
| Translation: | |
| MEGRRFAAVLILTICMLAPGTGRPSVCNKPADKGPC | (SEQ ID NO:86) |
| TGSEKRFYFSTYHNECRTFKYGGCEGNGNNFIHVYN | |
| CRRTCVYPPG | |
| Toxin Sequence: | |
| Arg-Xaa3-Ser-Val-Cys-Asn-Lys-Xaa3- | (SEQ ID NO:87) |
| Ala-Asp-Lys-Gly-Xaa3-Cys-Thr-Gly- | |
| Ser-Xaa1-Lys-Arg-Phe-Xaa5-Phe-Ser- | |
| Thr-Xaa5-His-Asn-Xaa1-Cys-Arg-Thr- | |
| Phe-Lys-Xaa5-Gly-Gly-Cys-Xaa1-Gly- | |
| Asn-Gly-Asn-Asn-Phe-Ile-His-Val- | |
| Xaa5-Asn-Cys-Arg-Arg-Thr-Cys-Val- | |
| Xaa5-Xaa3-Xaa3-# | |
| Name: ShK magus | |
| Species: magus | |
| Isolated: Yes | |
| Cloned: No | |
| Toxin Sequence: | |
| Arg-Xaa3-Ser-Val-Cys-Asn-Lys-Xaa3- | (SEQ ID NO:88) |
| Ala-Asp-Lys-Gly-Xaa3-Cys-Ala-Gly- | |
| Ser-Xaa1-Lys-Arg-Phe-Xaa5-Phe-Ser- | |
| Thr-Xaa5-His-Asn-Xaa1-Cys-Arg-Thr- | |
| Phe-Lys-Xaa5-Gly-Gly-Cys-Xaa1-Gly- | |
| Asn-Gly-Asn-Asn-Phe-Ile-His-Val- | |
| Xaa5-Asn-Cys-Arg-Arg-Thr-Cys-Val- | |
| Xaa5-Xaa3-Met-\hat{ } | |
| Name: ShK str-1 | |
| Species: striatus | |
| Isolated: Yes | |
| Cloned: No | |
| Toxin Sequence: | |
| Lys-Asp-Arg-Xaa3-Ser-Leu-Cys-Asp- | (SEQ ID NO:90) |
| Leu-Xaa3-Ala-Asp-Ser-Gly-Ser-Gly- | |
| Thr-Lys-Ala-Xaa1-Lys-Arg-Ile-Xaa5- | |
| Xaa5-Asn-Ser-Ala-Arg-Lys-Gln-Cys- | |
| Leu-Arg-Phe-Asp-Xaa5-Thr-Gly-Gln- | |
| Gly-Gly-Asn-Xaa1-Asn-Asn-Phe-Arg- | |
| Arg-Thr-Xaa5-Asp-Cys-Gln-Arg-Thr- | |
| Cys-Leu-Xaa5-Thr-\hat{ } | |
| Name: ShK str-2 | |
| Species: striatus | |
| Isolated: Yes | |
| Cloned: No | |
| Toxin Sequence: | |
| Gly-Arg-Xaa3-Lys-Asp-Arg-Xaa3-Ser- | (SEQ ID NO:91) |
| Xaa5-Cys-Asn-Leu-Xaa3-Ala-Asp-Ser- | |
| Gly-Ser-Gly-Thr-Lys-Xaa3-Xaa1-Gln- | |
| Arg-Ile-Xaa5-Xaa5-Asn-Ser-Ala-Lys- | |
| Lys-Gln-Cys-Val-Thr-Phe-Thr-Xaa-Asn- | |
| Gly-Lys-Gly-Gly-Asn-Gly-Asn-Asn-Phe- | |
| Ser-Arg-\hat{ } | |

Xaa1 is Glu or γ-carboxy-Glu
Xaa2 is Gln or pyro-Glu
Xaa3 is Pro or hydroxy-Pro
Xaa4 is Trp or bromo-Trp
Xaa5 is Tyr, ¹²⁵I-Tyr mono-iodo-Tyr, di-iodo-Tyr, O-sulpho-Tyr or O-phospho-Tyr
Xaa6 is Val, Ala, Asp or Gly
\hat{ } is free carboxyl or amidated C-terminus, preferably free carboxyl
H is free carboxyl or amidated C-terminus, preferably amidated

TABLE 2
Alignment of Conkunitzin Mature Peptide Sequences (Group 1)

| | |
|---|---|
| | 10 20 30 40 50 |
| C1757 | -SRRQD---LCSLPAEKGPCLAYMP------RFYFDWDSKLCKVFIYGGC |
| C1758 | -SRRQD---LCSLPAEKGPCLAYMP------RFYFDWDSKLCKVFIYGGC |
| Shk magus | ------RPSVCNKPADKGPCAGSEK------RFYFSTYHNECRTFKYGGC |
| C1687 | ------RPSVCNKPADKGPCTGSEK------RFYFSTYHNECRTFKYGGC |
| ShKm1 | ------RPSVCNKPADKGPCTGSEK------RFYFSTYHNECRTFKYGGC |
| C1708 | -SRRKARPSVCNLPPERGPCAGSER------RVYFDSTSKQCKMFTYGGC |
| C1790 | -SRLRDQLNRCKQPKVTGPCKGAFR------RYYFDTDVRNCKQFTYGGC |
| DI085 | --GGDD---RCTLQLSHGTCPGYFP------RWFYDSASGQCQSFIYSGC |
| C1705 | -AKPKSRPSYCNLPAERGPCEGSEK------RYYFDSYRTQCRLFKYGGC |
| C1704 | TLIPRGRPSECNLRKDSGSCTRSEQSTKSEQRYYFSTYYNECRNFTYSGC |
| C1741 | TLIPRGRPSYCNLRKDSGSCTRSEQSTESEYRYYFSTHYNECRMFTYKGC |
| | 60 70 |
| C1757 | QGNDNNFHSSTDCYDTCWFE-\hat{ } (SEQ ID NO:91) |
| C1758 | QGNDNNFHSSSDCYDTCWFE-\hat{ } (SEQ ID NO:92) |
| Shk magus | EGNGNNFIHVYNCRRTCVYPM\hat{ } (SEQ ID NO:3) |
| C1687 | EGNGNNFIHVYNCRRTCVYPP# (SEQ ID NO:93) |
| ShKm1E | GNGNNFIHVYNCRRTCVYPP# (SEQ ID NO:94) |
| C1708 | GGNGNNFKRAHDCLKMCVYSV# (SEQ ID NO:95) |
| C1790 | RGNDNNFLTYDDCNATCGPK-\hat{ } (SEQ ID NO:96) |
| DI085 | KGNANNFLSEEECRQACVSR-\hat{ } (SEQ ID NO:97) |
| C1705 | EGNRNNFIRVYDCRKKCLHA-# (SEQ ID NO:98) |
| C1704 | GGNRNNFIHMYDCRRKCVYTV# (SEQ ID NO:99) |
| C1741 | GGNRNNFIHMHDCRQKCVYTV# (SEQ ID NO:100) |

TABLE 3
Alignment of Conkunitzin Mature Peptide Sequences (Group 2)

| | |
|---|---|
| | 10 20 30 40 50 |
| ShK str-1 | ----KDRPSLCDLPADSGSGTKAEKRIYYNSARKQCLRFDYTGQ--GGN- |
| C1716 | TLLPRDRPSLCDLPADSGSGTKAEKRIYYNSARKQCLRFDYTGQ--GGN- |
| Conkunitzin S1 | TLLPRDRPSLCDLPADSGSGTKAEKRIYYNSARKQCLRFDYTGQ--GON- |
| C1742 | TLIPRDRPSYCNLPADSGSGTNREQRIYYNSARKQCLTFPYKGK--GGN- |
| ShK str-2 | -GRPKDRPSYCNLPADSGSGTKPEQRIYYNSAKKQCVTFTYNGK--GGN- |
| C1722 | -ARPKDRPSYCNLPADSGSGTKPEQRIYYNSAKKQCVTFTYNGK--GON- |
| C1736 | -ARPKDRPSYCNLPADSGSGTKPEQRIYYNSARKQCLTFTYNGK--GGN- |
| C1679 | -ARPKDRPSYCNLPADSGSGTKPEQRIYYNSAREQCLTFTYNGK--GGN- |
| C1723 | -ARPKDRPSYCNLPADSGSGTKPEQRIYYNSAKKQCVTFTYSGK--GGN- |
| C1726 | -AKPKSRPSYCNLPADSGSGTKSEKRIYYNSARKQCLGFTYHGE--GGN- |
| C1710 | -AKPKSRPSYCNLPADSGSGTKSEKRIYYNSARKQCLRFTYHGE--GGN- |
| C1733 | -AKPKSRPSYCNLPADSGSGTKSEKRIYYNSARKQCLRFTYHGE--GGN- |
| C1689 | -AKPKSRPSYCNLPADSGSGTKSEKRIYYNSARKQCLRFTYHGE--GGN- |
| C1728 | -AKPKSRPSYCNLPADSGSGTKSEKRIYYNSARKQCLRFTYHGEREGPNX |
| | 60 |
| ShK str-1 | ENNFRRTYDCQRTCLYT--\hat{ } (SEQ ID NO:1) |
| C1716 | ENNFRRTYDCQRTCLYT--# (SEQ ID NO:101) |
| Conkunitzin S1 | ENNPRRTYDCQRTCLYT--# (SEQ ID NO:102) |
| C1742 | ANNFSRTNDCRRTCQYPAA# (SEQ ID NO:103) |
| ShK str-2 | GNNFSR------------- (SEQ ID NO:2) |
| C1722 | GNNFSRTNDCRQTCQYPV-# (SEQ ID NO:104) |
| C1736 | ENKFIHTYDCRRTCQYTV-\hat{ }(SEQ ID NO:105) |
| C1679 | ENNFIHTYDCRRTCQYTV-\hat{ }(SEQ ID NO:106) |
| C1723 | GNNFSRTNDCRQTCQYPA-# (SEQ ID NO:107) |
| C1726 | ANNFLHTFDCQHTCLYTV-\hat{ }(SEQ ID NO:108) |
| C1710 | ANNFLHTFDCQHTCLYTV-\hat{ }(SEQ ID NO:109) |
| C1733 | ANNFLHTFDCQHTCLYTV-\hat{ }(SEQ ID NO:110) |
| C1689 | ANNFLHTFDCQHTCLYTV-\hat{ }(SEQ ID NO:111) |
| C1728 | KNNFLHTFDCQHTCLYTV-\hat{ }(SEQ ID NO:112) |

TABLE 4
Alignment of Conkunitzin Mature Peptide Sequences (Group 3)

| | |
|---|---|
| | 10 20 30 40 50 |
| C1769 | -FRLRGSRAEECSRSEALAKCYLPKDPGPCKAWKSRYYFNRYLNTCQEFS |
| C1768 | -FRLRGSPAEQCSRSEALAKCYLPKDPGPCKAWKSRYYFNRYLNTCQEFS |
| C1760 | -SKRQGPPTNKCSRDVALSKCYQPRDPGPCYASFRMYYFNRYINTCEKFT |
| C1756 | -SRRQGPPTNKCSRDVALSKCYQPRDPGPCYALLERYYFNRYINTCEKFT |
| C1754 | ASRRQGPPTNKCSRDVALSKCYQPRDPGPCNAFFEKYYFNRYINTCEKFT |
| | 60 70 80 90 100 |
| C1769 | YGGCRGNANRFDTMEDCQECCLLSVCRQPAEPGLCKAYMERYYFDMDSYD |
| C1768 | YGGCRGNANRFDTMEDCQECCLLSVCRQPAEPGLCKAYMERYYFDMDSYD |
| C1760 | YGGCQGNDNRFNTVEDCLGCCQLSVCRQPAEPGLCKGYFPRHYFDMDSYE |
| C1756 | YGGCQGNDNRFNTMEDCLGCCQLSVCRQPAEPGLCRGYFLRHYFDMDFNE |
| C1754 | YGGCQGNDNRFNTVEDCLGCCLLSVCRQPAETGPCRGYFPKYYFDMDSYE |
| | 110 120 |
| C1769 | CKPFIYGGCNGNDNNFLTYIECYGRCGPE\hat{ } (SEQ ID NO:113) |
| C1768 | CKPFIYGGCNGNDNNFLTYIECYGRCGPE\hat{ } (SEQ ID NO:114) |
| C1760 | CKTFIYGGCGGNDNNFLTDGECQGRCGPE\hat{ } (SEQ ID NO:115) |
| C1756 | CKEFIYGGCGGNDNNFLTDEECQGRCGPE\hat{ } (SEQ ID NO:116) |
| C1754 | CKQFIYGGCGGNDNNFGTVEECQGRCGPK\hat{ } (SEQ ID NO:117) |

TABLE 5
Conkunitzin D1008 (Group 4)

| | |
|---|---|
| | 10 20 30 40 50 |
| DI008 | ENTCEQPPEPGRCEGYYRRFFYNWRSGQCESFVYGGCGGNDNNFRTNAEC |
| | 60 70 80 90 100 |
| DI008 | EAACSSTNTYAISENTCEQPAEQGRCTEYVVRFFYNWKSDKCEPFVYGGC |
| | 110 120 130 140 150 |
| DI008 | GGNSNSFLTITECETACSSGNTYEDVCQLPADRGVCSAHLHMFYYNSSSG |
| | 160 170 180 190 200 |
| DI008 | QCHQFIYRGCGGNGNKFSTMRECQAKCSRGVKYSKVLPTKQWKFFRRNEP |
| D1008 | RYLLA\hat{ } (SEQ ID NO:118) |

### Example 7

### Biological Activity and Electrophysiological Characterization

ICV administration of conkunitzin ShK str-1 resulted in a dose-dependent increase in the percentage of animals displaying seizures. In general, ICV administration of conkunitzin ShK str-1 produced spastic running followed by tonic extension seizures. The ED₅₀ for ICV conkunitzin ShK str-1 was determined to be 0.96 nmol/mouse (95% confidence limits: 0.29 to 2.00 nmol). In contrast, IP administration of conkunitzin ShK str-1 (3 nmol/mouse) was without effect.

Conkunitzin ShK str-1 has been tested on the Shaker K⁺ channel and an inhibition of channel conductance was observed.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Atherton, E. and Sheppard, R. C. (1989). Solid-Phase Peptide Synthesis, A Practical Approach, IRL Press, Oxford.

Backes, B. J. and Ellman, J. A. (1999). J Org Chem Soc 64:2322-2330.

Barber, M. et al. (1982). Anal. Chem. 54:645A-657A.

Bamay, G. et al. (2000). J. Med. Chem.

Bitan, G. et al. (1997). J. Peptide Res. 49:421-426.

Bodansky et al. (1966). Chem. Ind. 38:1597-98.

Bulaj, G. et al. (2001). Biochemistry 40:13201-13208.

Cartier, G. E. et al. (1996). J. Biol. Chem. 271:7522-7528.

Colledge, C. J. et al. (1992). Toxicon 30:1111-1116.

Craik, D. J. et al. (1991). Toxicon 39:43-60.

Cruz, L. J. et al. (1987). J. Biol. Chem. 260:9280-9288.

Dodd, J. et al. (1983). J. Physiol. 334:255-268.

Duda Jr, T. F. et al. (2001). Biol. J. Linnean Soc. 73:391-409.

Espiritu, D. J. D. et al. (2001). Toxicon 39:1899-1916.

Gobbo, M. et al. (1992). Int. J. Pept. Protein Res. 40:51-54.

Haack, J. A. et al. (1990). J. Biol. Chem. 265:6025-6029.

Hammerland, L. G. et al. (1992). Eur. J. Pharmacol. 226:239-244.

Horiki, K. et al. (1978). Chemistry Letters 165-68.

Hubry, V. et al. (1994). Reactive Polymers 22:231-241.

Jacobsen, R. et al., (1997). J. Biol. Chem. 272:22531-22537.

Jacobsen, R. et al. (1998). J. Peptide Res. 51:173-179.

Jacobsen, R. B. et al. (2000). J. Biol. Chem. 275:24639-24644.

Kaiser et al. (1970). Anal. Biochem. 34:595.

Kapoor (1970). J. Pharm. Sci. 59:1-27.

Lowry, O. et al. (1951). J Biol. Chem. 193:265.

Litchfield, J. and Wilcoxon, F. (1949). J Pharmacol Exp Ther 96:99-113.

Mena, E. E. et al. (1990). Neurosci. Lett. 118:241-244.

Methfessel, C. et al. (1986). Pflugers Arch. 407:577-588.

Methoden der Organischen Chemie (Houben-Weyl): Synthese von Peptiden, E. Wunsch (Ed.), Georg Thieme Verlag, Stuttgart, Ger. (1974).

Nishiuchi, Y. et al. (1993). Int. J. Pept. Protein Res. 42:533-538.

Nguyen, T.-D. et al. (2000). J. Neurosci. Res. 60:175-183.

Olivera, B. M. et al. (1985). Science 230:1338-1343.

Olivera, B. M. et al. (1990). Science 249:257-263.

Olivera, B. M. (1997). Mol. Biol. Cell 8:2101-2109.

Olivera, B. M. et al. (1999). Ann. N.Y. Acad. Sci. 870:223-237.

Ornstein, et al. (1993). Biorganic Medicinal Chemistry Letters 3:43-48.

Piscano, A. et al. (1993). Glycobiology 3:429-435.

Remington's Pharmaceutical Sciences, 18th Ed., Mack Publishing Co., Easton, Pa. (1990).

Rivier, J. R. et al. (1978). Biopolymers 17:1927-38.

Rivier, J. R. et al. (1987). Biochem. 26:8508-8512.

Sambrook, J. et al. (1989). Molecular Cloning. A Laboratory Manual, 2nd Ed., Cold Spring Harbor Laboratory, Cold Spring Harbor, N.Y.

Schroder & Lubke (1965). The Peptides 1:72-75, Academic Press, NY.

Shin, Y. et al. (1999). J Am Chem Soc 121:11684-11689.

Shon, K-J. et al. (1998). J. Biol. Chem. 273:33-38.

Sole, N. A. and Barany, G. (1992). J Org Chem 57:5399-5403.

Stewart and Young, Solid-Phase Peptide Synthesis, Freeman & Co., San Francisco, Calif. (1969).

Stuhmer, W. (1992) Meth. Enzymol. 207, 319-339

Terlau, H. et al. (1996). Nature 381:148-151.

Terlau, H. et al. (1999). J. Gen. Physiol. 114:125-140.

Van de Steen et al. (1998). Crit. Rev. Biochem. Mol. Biol. 33:151-208.

Woodward, S. R. et al. (1990). EMBO J. 1:1015-1020.

Yoshikami, D. et al. (1989). Ann. N.Y. Acad. Sci. 560:230-248.

Zhou L. M., et al. (1996). J. Neurochem. 66:620-628.

U.S. patent application Ser. No. 09/910,009

U.S. Pat. No. 3,842,067.

U.S. Pat. No. 3,862,925.

U.S. Pat. No. 3,972,859.

U.S. Pat. No. 4,105,603.

U.S. Pat. No. 4,447,356.

U.S. Pat. No. 4,569,967.

U.S. Pat. No. 5,364,769.

U.S. Pat. No. 5,514,774.

U.S. Pat. No. 5,534,615.

U.S. Pat. No. 5,550,050.

U.S. Pat. No. 5,591,821.

U.S. Pat. No. 5,633,347.

U.S. Pat. No. 5,635,347.

U.S. Pat. No. 5,844,077.

U.S. Pat. No. 5,889,147.

U.S. Pat. No. 6,077,934.

PCT Published Application WO 92/19125.

PCT Published Application WO 94/25503.

PCT Published Application WO 95/01203.

PCT Published Application WO 96/02286.

PCT Published Application WO 96/02646.

PCT Published Application WO 96/05452.

PCT Published Application WO 96/11698.

PCT Published Application WO 96/40871.

PCT Published Application WO 96/40979.

PCT Published Application WO 97/12635.

PCT Published Application WO 02/07678.

What is claimed is:

1. A peptide selected from the group consisting of:
(i) a peptide comprising an amino acid sequence selected from the group consisting of the amino acid sequences set forth in SEQ ID NO:6, SEQ ID NO:9, SEQ ID NO:12, SEQ ID NO:15, SEQ ID NO:18, SEQ ID NO:21, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, SEQ ID NO:72, SEQ ID NO:75, SEQ ID NO:78, SEQ ID NO:81, SEQ ID NO:84, SEQ ID NO:87, SEQ ID NO:88, SEQ ID NO:89 and SEQ ID NO:90;
(ii) a peptide comprising an amino acid sequence selected from the group consisting of the amino acid sequences set forth in SEQ ID NO:3, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99 and SEQ ID NO:100;
(iii) a peptide comprising an amino acid sequence selected from the group consisting of the amino acid sequences set forth in SEQ ID NO:1, SEQ ID NO:2, SEQ SEQ ID NO:101, ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111 and SEQ ID NO:112;
(iv) a peptide comprising an amino acid sequence selected from the group consisting of the amino acid sequences set forth in SEQ ID NO:113, SEQ ID NO:114, SEQ ID NO:115, SEQ ID NO:116 and SEQ ID NO:117;
(v) a peptide comprising the amino acid sequence set forth in SEQ ID NO:118;
(vi) propeptide comprising an amino acid sequence selected from the group consisting of the amino acid sequences set forth in SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:74, SEQ ID NO:77, SEQ ID NO:60, SEQ ID NO:83 and SEQ ID NO:86; and
(vii) a derivative of (a) the peptide of (i), (ii), (iii), (iv) or (v) or (b) the propeptide of (vi).

2. The peptide of claim 1, wherein said derivative is the peptide or propeptide in which Arg residues may be substituted by Lys, omithine, homoargine, nor-Lys, N-methyl-Lys, N,N-dimethyl-Lys, N,N,N-trimethyl-Lys or any synthetic basic amino acid; the Lys residues may be substituted by Arg, omithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated; the halogen may be iodo, chloro, fluoro or bromo; preferably iodo for halogen substituted-Tyr and bromo for halogen-substituted Trp; the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂ₙ₊₂ up to and including n=8; the Leu residues may be substituted with Leu (D); the Glu residues may be substituted with Gla; the Met residues may be substituted with norleucine (Nle); the Cys residues may be in D or L configuration and may optionally be substituted with homocysteine (D or L).

3. The peptide of claim 1, wherein the peptide is the propeptide comprising an amino acid sequence selected from the group consisting of the amino acid sequences set forth in SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:74, SEQ ID NO:77, SEQ ID NO:60, SEQ ID NO:83 and SEQ ID NO:86.

4. A nucleic acid encoding the propeptide of claim 3.

5. The nucleic acid of claim 4 comprising a nucleotide sequence selected form the group of nucleotide sequences set forth in SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, SEQ ID NO:34, SEQ ID NO:37, SEQ ID NO:40, SEQ ID NO:43, SEQ ID NO:46, SEQ ID NO:49, SEQ ID NO:52, SEQ ID NO:55, SEQ ID NO:58, SEQ ID NO:61, SEQ ID NO:64, SEQ ID NO:67, SEQ ID NO:70, SEQ ID NO:73, SEQ ID NO:76, SEQ ID NO:79, SEQ ID NO:82 and SEQ ID NO:85.

6. A method for regulating the flow of potassium through potassium channels in an individual in need thereof which comprises administering a therapeutically effective amount of a conkunitzin.

7. The method of claim 6, wherein said individual in need thereof (i) suffers from a disorder selected from the group consisting of multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction and disorders resulting from defects in neurotransmitter release (such as Eaton-Lambert syndrome) or (ii) needs a reversal of the actions of curare and other neuromuscular blocking drugs.

8. The method of claim 7, wherein said disorder is a demyelinating disease.

9. The method of claim 6, wherein said conkunitzin is selected from the group consisting of:
(i) a peptide comprising an amino acid sequence selected from the group consisting of the amino acid sequences set forth in SEQ ID NO:6, SEQ ID NO:9, SEQ ID NO:12, SEQ ID NO:15, SEQ ID NO:18, SEQ ID NO:21, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, SEQ ID NO:72, SEQ ID NO:75, SEQ ID NO:78, SEQ ID NO:81, SEQ ID NO:84, SEQ ID NO:87, SEQ ID NO:88, SEQ ID NO:89 and SEQ ID NO:90;
(ii) a peptide comprising an amino acid sequence selected from the group consisting of the amino acid sequences set forth in SEQ ID NO:3, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99 and SEQ ID NO:100;
(iii) a peptide comprising an amino acid sequence selected from the group consisting of the amino acid sequences set forth in SEQ ID NO:1, SEQ ID NO:2, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111 and SEQ ID NO:112;
(iv) a peptide comprising an amino acid sequence selected from the group consisting of the amino acid sequences set forth in SEQ ID NO:113, SEQ ID NO:114, SEQ ID NO: 115, SEQ ID NO:116 and SEQ ID NO:117;
(v) a peptide comprising the amino acid sequence set forth in SEQ ID NO:118; and
(vi) a derivative of the peptide of (i), (ii), (iii), (iv) or (v).

10. The method of claim 9, wherein said derivative is the peptide in which Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, omithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated; the halogen may be iodo, chloro, fluoro or bromo; preferably iodo for halogen substituted-Tyr and bromo for halogen-substituted Trp; the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂ₙ₊₂ up to and including n=8; the Leu residues may be substituted with Leu (D); the Glu residues may be substituted with Gla; the Met residues may be substituted with norleucine (Nle); the Cys residues may be in D or L configuration and may optionally be substituted with homocysteine (D or L).

11. A method of identifying compounds that mimic the therapeutic activity of a conkunitzin, comprising the steps of: (a) conducting a biological assay on a test compound to determine the therapeutic activity; and (b) comparing the results obtained from the biological assay of the test compound to the results obtained from the biological assay of a conkunitzin.

12. The method of claim 11, wherein said conkunitzin is selected from the group consisting of:
(i) a peptide comprising an amino acid sequence selected from the group consisting of the amino acid sequences set forth in SEQ ID NO:6, SEQ ID NO:9, SEQ ID NO:12, SEQ ID NO:15, SEQ ID NO:18, SEQ ID NO:21, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, SEQ ID NO:72, SEQ ID NO:75, SEQ ID NO:78, SEQ ID NO:81, SEQ ID NO:84, SEQ ID NO:87, SEQ ID NO:88, SEQ ID NO:89 and SEQ ID NO:90;
(ii) a peptide comprising an amino acid sequence selected from the group consisting of the amino acid sequences set forth in SEQ ID NO:3, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99 and SEQ ID NO:100;
(iii) a peptide comprising an amino acid sequence selected from the group consisting of the amino acid sequences set forth in SEQ ID NO:1, SEQ ID NO:2, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111 and SEQ ID NO:112;
(iv) a peptide comprising an amino acid sequence selected from the group consisting of the amino acid sequences set forth in SEQ ID NO:113, SEQ ID NO:114, SEQ ID NO:115, SEQ ID NO:116 and SEQ ID NO:117;
(v) a peptide comprising the amino acid sequence set forth in SEQ ID NO:118; and
(vi) a derivative of the peptide of (i), (ii), (iii), (iv) or (v).

13. The method of claim 12, wherein said derivative is the peptide in which Arg residues may be substituted by Lys, omithine, homoargine, nor-Lys, N-methyl-Lys, N,N-dimethyl-Lys, N,N,N-trimethyl-Lys or any synthetic basic amino acid; the Lys residues may be substituted by Arg, omithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated; the halogen may be iodo, chloro, fluoro or bromo; preferably iodo for halogen substituted-Tyr and bromo for halogen-substituted Trp; the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂₊₂ up to and including n=8; the Leu residues may be substituted with Leu (D); the Glu residues may be substituted with Gla; the Met residues may be substituted with norleucine (Nle); the Cys residues may be in D or L configuration and may optionally be substituted with homocysteine (D or L).
